(19) United States
(12) Patent Application Publication
(10) Pub. No.: US 2025/0248879 A1
(43) Pub. Date: Aug. 7, 2025

(54) METHOD FOR OUTPUTTING REFERENCE IMAGE ON BASIS OF SENSING INFORMATION OF WEARABLE DEVICE, AND ELECTRONIC DEVICE WHICH CARRIES OUT SAME

(71) Applicant: Samsung Electronics Co., Ltd., Suwon-si (KR)

(72) Inventors: Hyunkyung SONG, Suwon-si (KR); Seungjoon LEE, Suwon-si (KR); Sugyeong KIM, Suwon-si (KR)

(21) Appl. No.: 19/093,821

(22) Filed: Mar. 28, 2025

Related U.S. Application Data
(63) Continuation of application No. PCT/KR2023/015214, filed on Oct. 4, 2023.

(30) Foreign Application Priority Data
Oct. 5, 2022 (KR) 10-2022-0126834

(51) Int. Cl. A61H 3/00; A61H 1/02; G16H 40/63

(52) U.S. Cl. CPC A61H 3/00; G16H 40/63; A61H 1/0244; A61H 2201/5007; A61H 2201/5043; A61H 2230/065; A61H 2230/625

(57) ABSTRACT

An electronic device may: obtain sensing information of a first time with respect to a motion of a user wearing a wearable device; determine a target index with respect to the motion on the basis of the sensing information; determine a reference index of the first time for a reference image; determine whether the target index corresponds to the reference index; and control outputting of the reference image with respect to a target time, which is later than the first time, on the basis of whether the target index corresponds to the reference index.

### CROSS-REFERENCE TO RELATED APPLICATIONS

This application is a continuation application of International Application No. PCT/KR2023/015214 designating the United States, filed on Oct. 4, 2023, in the Korean Intellectual Property Receiving Office and claiming priority to Korean Patent Application No. 10-2022-0126834, filed on Oct. 5, 2022, the disclosures of which are all hereby incorporated by reference herein in their entireties.

### BACKGROUND

### Technical Field

Certain example embodiments may relate to a method of controlling an output of an image based on sensing information obtained from a wearable device to be worn by a user.

### Background Art

A change to aging societies has contributed to a growing number of people who experience walking inconvenience and pain from reduced muscular strength or joint problems due to aging. Thus, there is a growing interest in walking assist devices for assisting elderly users or patients with reduced muscular strength or joint problems to walk with less effort, and/or to assist persons with exercise.

### SUMMARY

According to an example embodiment, an electronic device may include a communication module, comprising communication circuitry, configured to exchange data with an external device, and at least one processor, comprising processing circuitry, individually and/or collectively configured to control the electronic device to perform an operation of obtaining first sensing information with respect to a first motion of a user wearing a wearable device, wherein the first sensing information is measured at a first time, an operation of determining a first target index for the first motion based on the first sensing information, an operation of determining a first reference index of a reference image output at the first time, an operation of determining whether the first target index corresponds to the first reference index, and an operation of controlling an output of the reference image at a target time after the first time based on whether the first target index corresponds to the first reference index.

According to an example embodiment, a method, performed by an electronic device, of outputting a reference image may include: an operation of obtaining first sensing information with respect to a first motion of a user wearing a wearable device, wherein the first sensing information is measured at a first time, an operation of determining a first target index for the first motion based on the first sensing information, an operation of determining a first reference index of a reference image output at the first time, an operation of determining whether the first target index corresponds to the first reference index, and an operation of controlling an output of the reference image at a target time after the first time based on whether the first target index corresponds to the first reference index.

### BRIEF DESCRIPTION OF DRAWINGS

FIG. 1 is a diagram illustrating a configuration of a system for providing a user with an exercise program according to an example embodiment.

FIG. 2 is a block diagram of an electronic device in a network environment, according to an example embodiment.

FIGS. 3A, 3B, 3C, and 3D are diagrams illustrating a wearable device, according to an example embodiment(s).

FIG. 4 is a diagram illustrating a wearable device communicating with an electronic device, according to an example embodiment.

FIGS. 5 and 6 are diagrams illustrating a torque output method of a wearable device, according to an example embodiment.

FIG. 7 is a flowchart of a method of controlling an output of a reference image based on first sensing information on a first motion of a user, according to an example embodiment.

FIG. 8 is a flowchart of a method of determining a first target index, according to an example embodiment.

FIG. 9 illustrates reference indices for each of a plurality of classification motions forming an exercise mode, according to an example embodiment.

FIG. 10 is a flowchart of a method of determining whether a first target index corresponds to a first reference index, according to an example embodiment.

FIG. 11 is a flowchart of a method of controlling an output of a reference image for a target sight when a first target index does not correspond to a first reference index, according to an example embodiment.

FIG. 12 is a flowchart of a method of determining a target output speed of a reference image when a first target index does not correspond to a first reference index, according to an example embodiment.

FIG. 13 illustrates a method of controlling an output of a reference image, according to an example embodiment.

FIG. 14 is a flowchart of a method of controlling an output of a second reference image corresponding to a target index pattern of a first target index, according to an example embodiment.

FIG. 15 is a method of controlling an output of a modified second reference image, according to an example embodiment.

FIG. 16 is a flowchart of a method of stopping outputting a reference image, according to an example embodiment.

FIG. 17 is a flowchart of a method of outputting a third reference image based on a second motion of a user after outputting a reference image is stopped, according to an example embodiment.

FIG. 18 is a flowchart of pausing outputting a reference image when a motion of a user is inaccurate, according to an example embodiment.

FIG. 19 is a flowchart of a method of outputting an additional reference image when a connection of a second wearable device is detected, according to an example embodiment.

FIG. 20 is a flowchart of a method of controlling outputting a reference image based on a heart rate of a user, according to an example embodiment.

FIG. 21 is a flowchart of a method of controlling an electronic device based on a target gesture input, according to an example embodiment.

### DETAILED DESCRIPTION

Hereinafter, various embodiments of the present disclosure will be described with reference to the accompanying drawings. However, this is not intended to limit the present disclosure to specific embodiments, and it should be understood that various modifications, equivalents, and/or alternatives of the embodiments of the present disclosure are included.

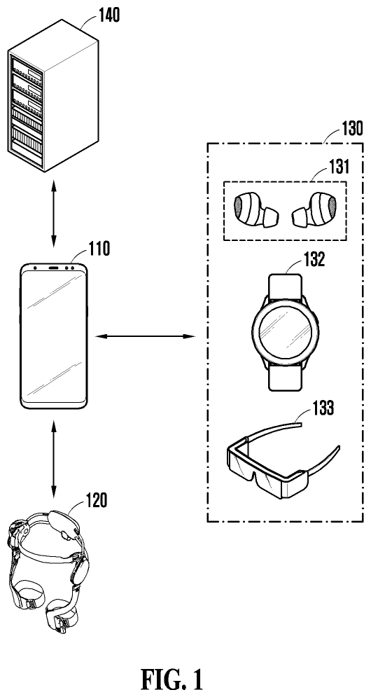

FIG. 1 is a diagram illustrating a configuration of a system for providing a user with a reference image, according to an embodiment.

According to an embodiment, a system 100 for providing a user with an exercise program may include an electronic device 110, a wearable device 120, an additional device 130, and a server 140.

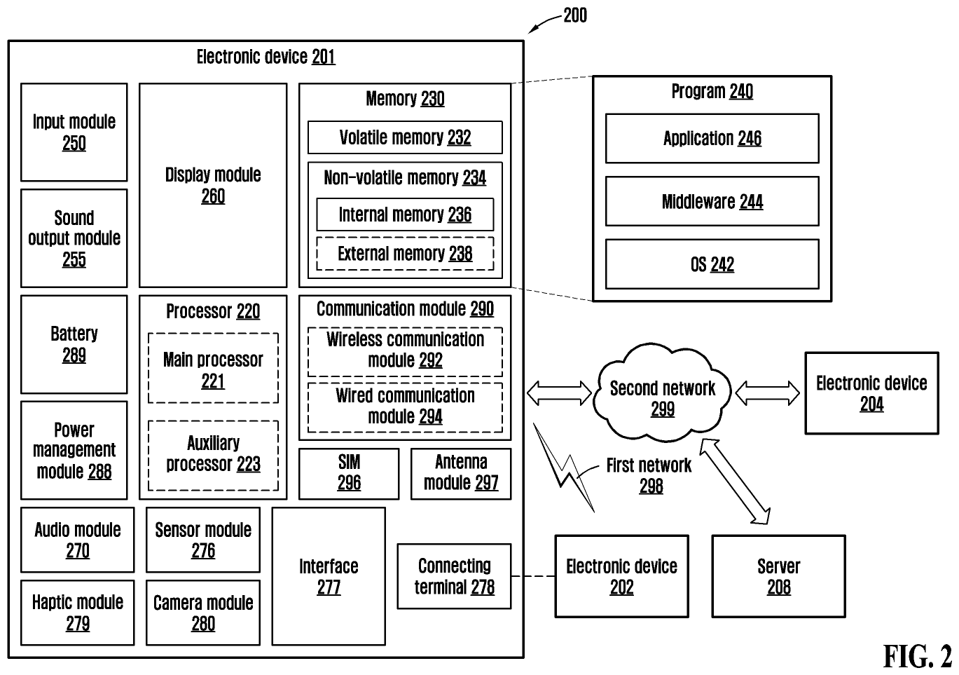

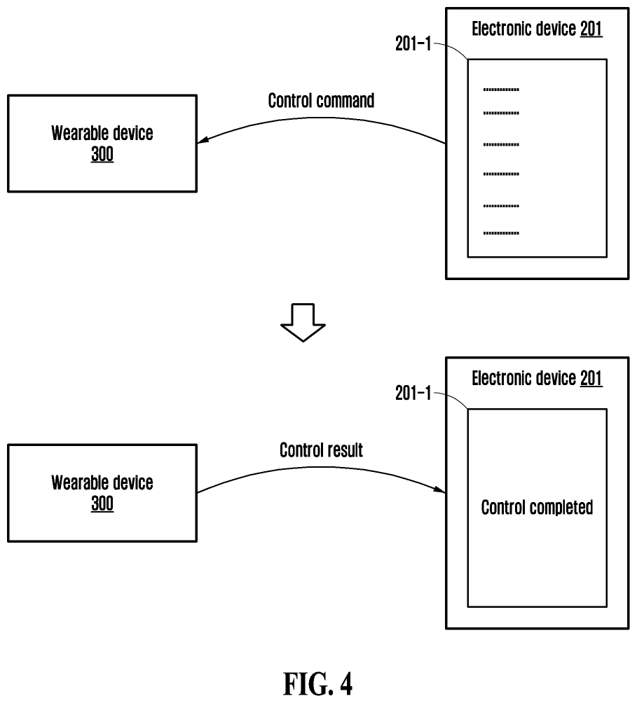

According to an embodiment, the electronic device 110 may be a user terminal that may be connected to the wearable device 120 using short-range wireless communication. For example, the electronic device 110 may transmit a control signal for controlling the wearable device 120 to the wearable device 120. The electronic device 110 will be described in detail below with reference to FIG. 2, and the transmission of a control signal will be described in detail below with reference to FIG. 4.

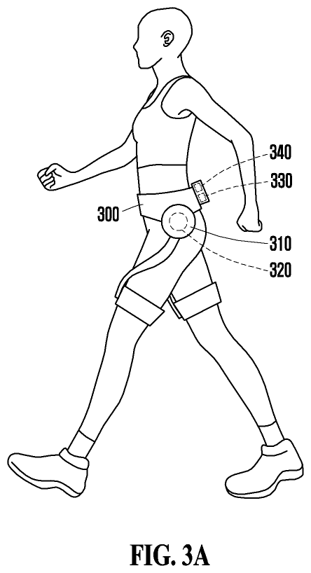

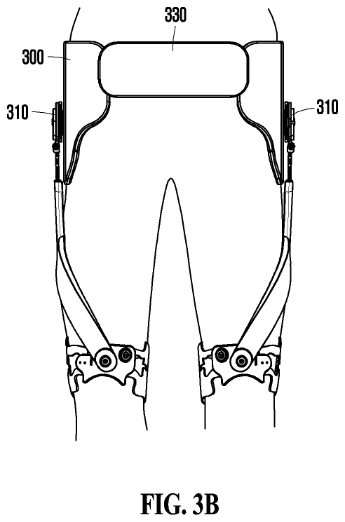

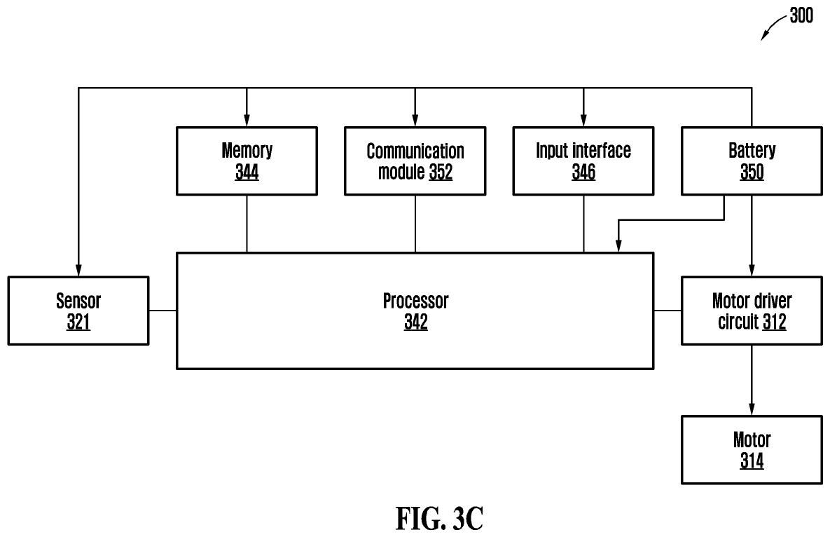

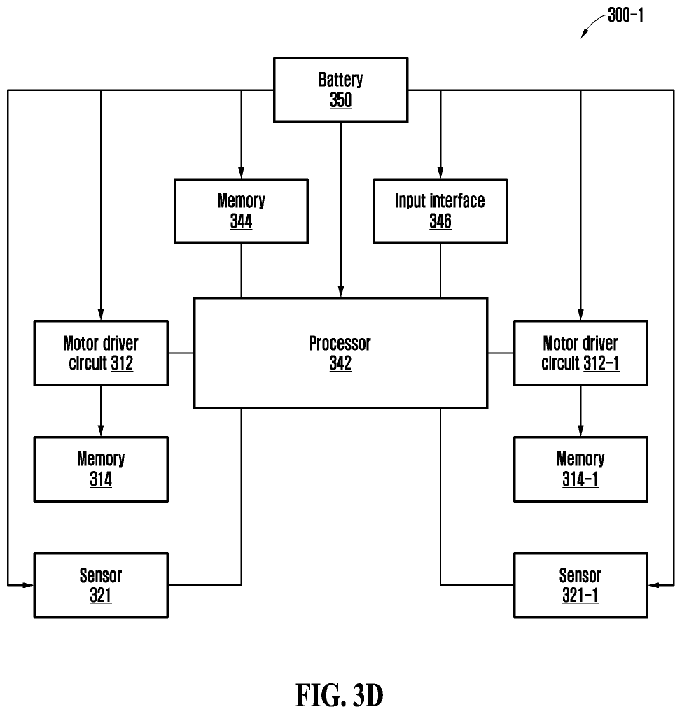

According to an embodiment, the wearable device 120 may provide a user wearing the wearable device 120 with an assistance force for assisting a motion (e.g., a gait or exercise in place) or a resistance force for impeding a motion. The resistance force may be provided to the user to assist the user in doing an exercise. The values of various control parameters (or robot parameters) used in the wearable device 120 may be controlled to control the assistance force or the resistance force output by the wearable device 120. The structure and driving method of the wearable device 120 will be described in detail below with reference to FIGS. 3A, 3B, 3C, 3D, 4, 5, and 6.

According to an example embodiment, the electronic device 110 may be connected to the additional device 130 (e.g., wireless earphones 131, a smartwatch 132, or smart glasses 133) using short-range wireless communication. For example, the electronic device 110 may output information indicating the state of the electronic device 110 or the state of the wearable device 120 to the user through the additional device 130. For example, feedback information with respect to a motion of the user wearing the wearable device 120 may be output through a haptic device, a speaker device, and a display device of the additional device 130.

According to an embodiment, the electronic device 110 may be connected to the server 140 using short-range wireless communication or cellular communication. For example, the server 140 may include a database in which information about a plurality of workout programs to be provided to a user through the wearable device 120 is stored. For example, the server 140 may manage a user account of the user of the electronic device 110 or the wearable device 120. The server 140 may store and manage a workout program performed by the user and a result of performance with respect to the workout program in link with the user account.

According to an embodiment, when the user performs the exercise mode while wearing the wearable device 120, the electronic device 110 may receive, from the wearable device 120, sensing information on a motion of the user. Each exercise mode may relate to a body motion to achieve a predetermined exercise goal. For example, an exercise mode of a step exercise type may include an exercise mode for symmetrically or alternately moving a left leg and a right leg, such as a fast feet mode, a lunge mode, a split 15 jack mode, a toe tap triceps mode, a knee up mode, a march step with twist mode, or a mountain climber mode. For example, an exercise mode of a non-step exercise type may include an exercise mode for moving a left leg and a right leg in the same direction, such as a squat mode, a narrow squat mode, a half squat mode, a deadlift mode, a single leg deadlift mode, a kickback mode, a bird dog mode, or a good morning mode.

According to an embodiment, the electronic device 110 may output a reference image related to the exercise mode of the user based on the sensing information. For example, the reference image may be an image that is produced in advance to instruct a posture of the exercise mode.

According to an embodiment, the electronic device 110 may calculate a motion repetition speed of the user based on the sensing information and may control an output speed of the reference image to correspond to the calculated repetition speed.

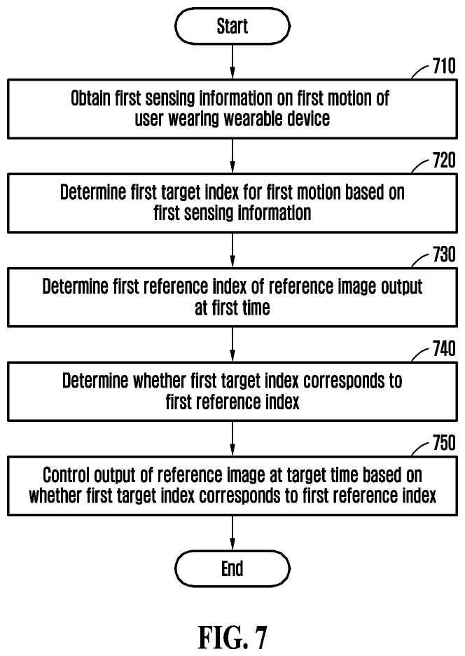

Hereinafter, a method of controlling an output of a reference image is further described with reference to FIGS. 7 to 21.

FIG. 2 is a block diagram of an electronic device in a network environment, according to an embodiment.

FIG. 2 is a block diagram of an electronic device 201 (e.g., the electronic device 110 of FIG. 1) in a network environment 200 according to an embodiment. Referring to FIG. 2, the electronic device 201 in a network environment 200 may communicate with an electronic device 202 via a first network 298 (e.g., a short-range wireless communication network), or at least one of an electronic device 204 or a server 208 via a second network 299 (e.g., a long-range wireless communication network). According to an embodiment, the electronic device 201 may communicate with the electronic device 204 via the server 208. According to an embodiment, the electronic device 201 may include a processor 220, a memory 230, an input module 250, a sound output module 255, a display module 260, an audio module 270, a sensor module 276, an interface 277, a connecting terminal 278, a haptic module 279, a camera module 280, a power management module 288, a battery 289, a communication module 290, a subscriber identification module (SIM) 296, or an antenna module 297. In some embodiments, at least one (e.g., the connecting terminal 278) of the above components may be omitted from the electronic device 201, or one or more other components may be added to the electronic device 201. In some embodiments, some (e.g., the sensor module 276, the camera module 280, or the antenna module 297) of the components may be integrated as a single component (e.g., the display module 260).

The processor 220 may execute, for example, software (e.g., a program 240) to control at least one other component (e.g., a hardware or software component) of the electronic device 201 coupled with the processor 220, and may perform various data processing or computation. According to an embodiment, as at least a portion of data processing or computation, the processor 220 may store a command or data received from another component (e.g., the sensor module 276 or the communication module 290) in a volatile memory 232, process the command or the data stored in the volatile memory 232, and store resulting data in a non-volatile memory 234. According to an embodiment, the processor 220 may include a main processor 221 (e.g., a central processing unit (CPU) or an application processor (AP)) or an auxiliary processor 223 (e.g., a graphics processing unit (GPU), a neural processing unit (NPU), an image signal processor (ISP), a sensor hub processor, or a communication processor (CP)) that is operable independently of, or in conjunction with the main processor 221. For example, when the electronic device 201 includes the main processor 221 and the auxiliary processor 223, the auxiliary processor 223 may be adapted to consume less power than the main processor 221 or to be specific to a specified function. The auxiliary processor 223 may be implemented as separate from, or as part of the main processor 221.

The auxiliary processor 223 may control at least some of functions or states related to at least one component (e.g., the display module 260, the sensor module 276, or the communication module 290) among the components of the electronic device 201, instead of the main processor 221 while the main processor 221 is in an inactive (e.g., sleep) state, or together with the main processor 221 while the main processor 221 is in an active state (e.g., executing an application). According to an embodiment, the auxiliary processor 223 (e.g., an ISP or a CP) may be implemented as a portion of another component (e.g., the camera module 280 or the communication module 290) that is functionally related to the auxiliary processor 223. According to an embodiment, the auxiliary processor 223 (e.g., an NPU) may include a hardware structure specified for artificial intelligence (AI) model processing. An artificial intelligence model may be generated by machine learning. Such learning may be performed, e.g., by the electronic device 201 where the artificial intelligence is performed, or via a separate server (e.g., the server 208). Learning algorithms may include, but are not limited to, e.g., supervised learning, unsupervised learning, semi-supervised learning, or reinforcement learning. The AI model may include a plurality of artificial neural network layers. An artificial neural network may include, for example, a deep neural network (DNN), a convolutional neural network (CNN), a recurrent neural network (RNN), a restricted Boltzmann machine (RBM), a deep belief network (DBN), a bidirectional recurrent deep neural network (BRDNN), a deep Q-network, or a combination of two or more thereof, but is not limited thereto. The artificial intelligence model may, additionally or alternatively, include a software structure other than the hardware structure.

Each “processor” herein includes processing circuitry, and/or may include multiple processors. For example, as used herein, including the claims, the term “processor” may include various processing circuitry, including at least one processor, wherein one or more of at least one processor, individually and/or collectively in a distributed manner, may be configured to perform various functions described herein. As used herein, when “a processor”, “at least one processor”, and “one or more processors” are described as being configured to perform numerous functions, these terms cover situations, for example and without limitation, in which one processor performs some of recited functions and another processor(s) performs other of recited functions, and also situations in which a single processor may perform all recited functions. Additionally, the at least one processor may include a combination of processors performing various of the recited/disclosed functions, e.g., in a distributed manner. At least one processor may execute program instructions to achieve or perform various functions.

The memory 230 may store various data used by at least one component (e.g., the processor 220 or the sensor module 276) of the electronic device 201. The various pieces of data may include, for example, software (e.g., the program 240) and input data or output data for a command related thereto. The memory 230 may include the volatile memory 232 or the non-volatile memory 234.

The program 240 may be stored as software in the memory 230, and may include, for example, an operating system (OS) 242, middleware 244, or an application 246. The input module 250 may receive a command or data to be used by another component (e.g., the processor 220) of the electronic device 201, from the outside (e.g., a user) of the electronic device 201. The input module 250 may include, for example, a microphone, a mouse, a keyboard, a key (e.g., a button), or a digital pen (e.g., a stylus pen).

The sound output module 255 may output sound signals to the outside of the electronic device 201. The sound output module 255 may include, for example, a speaker or a receiver. The speaker may be used for general purposes, such as playing multimedia or playing a record. The receiver may be used to receive an incoming call. According to an embodiment, the receiver may be implemented separately from the speaker or as a portion of the speaker.

The display module 260 may visually provide information to the outside (e.g., a user) of the electronic device 201. The display module 260 may include, for example, a display, a hologram device, or a projector and control circuitry to control a corresponding one of the display, hologram device, and projector. According to an embodiment, the display module 260 may include a touch sensor adapted to sense a touch, or a pressure sensor adapted to measure an intensity of a force incurred by the touch.

The audio module 270 may convert a sound into an electrical signal and vice versa. According to an embodiment, the audio module 270 may obtain the sound via the input module 250 or output the sound via the sound output module 255 or an external electronic device (e.g., an electronic device 202 such as a speaker or a headphone) directly or wirelessly connected to the electronic device 201.

The sensor module 276 may detect an operational state (e.g., power or temperature) of the electronic device 201 or an environmental state (e.g., a state of a user) external to the electronic device 201, and then generate an electrical signal or data value corresponding to the detected state. According to an embodiment, the sensor module 276 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 277 may support one or more specified protocols to be used for the electronic device 201 to be coupled with the external electronic device (e.g., the electronic device 202) directly (e.g., wired) or wirelessly. According to an embodiment, the interface 277 may include, for example, a high-definition multimedia interface (HDMI), a universal serial bus (USB) interface, a secure digital (SD) card interface, or an audio interface.

The connecting terminal 278 may include a connector via which the electronic device 201 may be physically connected to an external electronic device (e.g., the electronic device 202). According to an embodiment, the connecting terminal 278 may include, for example, an HDMI connector, a USB connector, an SD card connector, or an audio connector (e.g., a headphone connector).

The haptic module 279 may convert an electrical signal into a mechanical stimulus (e.g., a vibration or a movement) or electrical stimulus which may be recognized by a user via his tactile sensation or kinesthetic sensation. According to an embodiment, the haptic module 279 may include, for example, a motor, a piezoelectric element, or an electric stimulator.

The camera module 280 may capture a still image and moving images.

According to an embodiment, the camera module 280 may include one or more lenses, image sensors, ISPs, or flashes.

The power management module 288 may manage power supplied to the electronic device 201. According to an embodiment, the power management module 288 may be implemented as, for example, at least a portion of a power management integrated circuit (PMIC).

The battery 289 may supply power to at least one component of the electronic device 201. According to an embodiment, the battery 289 may include, for example, a primary cell that is not rechargeable, a secondary cell that is rechargeable, or a fuel cell.

The communication module 290 may support establishing a direct (e.g., wired) communication channel or a wireless communication channel between the electronic device 201 and the external electronic device (e.g., the electronic device 202, the electronic device 204, or the server 208) and performing communication via the established communication channel. The communication module 290 may include one or more communication processors that are operable independently from the processor 220 (e.g., the application processor) and support direct (e.g., wired) communication or wireless communication. According to an embodiment, the communication module 290 may include a wireless communication module 292 (e.g., a cellular communication module, a short-range wireless communication module, or a global navigation satellite system (GNSS) communication module) or a wired communication module 294 (e.g., a local area network (LAN) communication module, or a power line communication (PLC) module). A corresponding one of these communication modules may communicate with the external electronic device 204 via the first network 298 (e.g., a short-range communication network, such as Bluetooth™, wireless-fidelity (Wi-Fi) direct, or infrared data association (IrDA)) or the second network 299 (e.g., a long-range communication network, such as a legacy cellular network, a 5G network, a next-generation communication network, the Internet, or a computer network (e.g., a LAN or a wide area network (WAN))). These various types of communication modules may be implemented as a single component (e.g., a single chip), or may be implemented as multiple components (e.g., multiple chips) separate from each other. The wireless communication module 292 may identify and authenticate the electronic device 201 in a communication network, such as the first network 298 or the second network 299, using subscriber information (e.g., international mobile subscriber identity (IMSI)) stored in the SIM 296.

The wireless communication module 292 may support a 5G network after a 4G network, and a next-generation communication technology, e.g., a new radio (NR) access technology. The NR access technology may support enhanced mobile broadband (eMBB), massive machine type communications (mMTC), or ultra-reliable and low-latency communications (URLLC). The wireless communication module 292 may support a high-frequency band (e.g., a mmWave band) to achieve, e.g., a high data transmission rate. The wireless communication module 292 may support various technologies for securing performance on a high-frequency band, such as, e.g., beamforming, massive multiple-input and multiple-output (massive MIMO), full dimensional MIMO (FD-MIMO), an array antenna, analog beamforming, or a large scale antenna. The wireless communication module 292 may support various requirements specified in the electronic device 201, an external electronic device (e.g., the electronic device 204), or a network system (e.g., the second network 299). According to an embodiment, the wireless communication module 292 may support a peak data rate (e.g., 20 Gbps or more) for implementing eMBB, loss coverage (e.g., 164 dB or less) for implementing mMTC, or U-plane latency (e.g., 0.5 ms or less for each of downlink (DL) and uplink (UL), or a round trip of 1 ms or less) for implementing URLLC.

The antenna module 297 may transmit or receive a signal or power to or from the outside (e.g., the external electronic device) of the electronic device 201. According to an embodiment, the antenna module 297 may include an antenna including a radiating element including a conductive material or a conductive pattern formed in or on a substrate (e.g., a printed circuit board (PCB)). According to an embodiment, the antenna module 297 may include a plurality of antennas (e.g., array antennas). In such a case, at least one antenna appropriate for a communication scheme used in a communication network, such as the first network 298 or the second network 299, may be selected by, for example, the communication module 290 from the plurality of antennas. The signal or the power may be transmitted or received between the communication module 290 and the external electronic device via the at least one selected antenna. According to an embodiment, another component (e.g., a radio frequency integrated circuit (RFIC)) other than the radiating element may be additionally formed as a portion of the antenna module 297.

According to an embodiment, the antenna module 297 may form an mmWave antenna module. According to an example embodiment, the mmWave antenna module may include a PCB, an RFIC disposed on a first surface (e.g., a bottom surface) of the PCB or adjacent to the first surface and capable of supporting a designated a high-frequency band (e.g., the mm Wave band), and a plurality of antennas (e.g., array antennas) disposed on a second surface (e.g., a top or a side surface) of the PCB, or adjacent to the second surface and capable of transmitting or receiving signals in the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 201 and the external electronic device 204 via the server 208 coupled with the second network 299. Each of the external electronic devices 202 and 204 may be a device of a same type as, or a different type from, the electronic device 201. According to an embodiment, all or some of operations to be executed by the electronic device 201 may be executed at one or more of external electronic devices (e.g., the external electronic devices 202 and 204, or the server 208). For example, if the electronic device 201 should perform a function or a service automatically, or in response to a request from a user or another device, the electronic device 201, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 201. The electronic device 201 may provide the outcome, with or without further processing of the outcome, as at least part of a reply to the request. To that end, a cloud computing, distributed computing, mobile edge computing (MEC), or client-server computing technology may be used, for example. The electronic device 201 may provide ultra low-latency services using, e.g., distributed computing or mobile edge computing. In another embodiment, the external electronic device 204 may include an Internet-of-things (IoT) device. The server 208 may be an intelligent server using machine learning and/or a neural network. According to an embodiment, the external electronic device 204 or the server 208 may be included in the second network 299. The electronic device 201 may be applied to intelligent services (e.g., smart home, smart city, smart car, or healthcare) based on 5G communication technology or IoT-related technology.

The electronic device according to the embodiments disclosed herein may be one of various types of electronic devices. The electronic device may include, for example, a portable communication device (e.g., a smartphone), a computer device, a portable multimedia device, a portable medical device, a camera, a wearable device, or a home appliance device. According to an example embodiment of the disclosure, the electronic device is not limited to those described above.

It should be appreciated that various embodiments of the present disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related components. It is to be understood that a singular form of a noun corresponding to an item may include one or more of the things unless the relevant context clearly indicates otherwise. As used herein, “A or B,” “at least one of A and B,” “at least one of A or B,” “A, B or C,” “at least one of A, B and C,” and “at least one of A, B, or C,” may include any one of the items listed together in the corresponding one of the phrases, or all possible combinations thereof. Terms such as “Ist” and “2nd,” or “first” and “second” may be used to simply distinguish a corresponding component from other components, and do not limit the components in other aspects (e.g., importance or order). It is to be understood that if an element (e.g., a first element) is referred to, with or without the term “operatively” or “communicatively”, as “coupled with,” “coupled to,” “connected with,” or “connected to” another element (e.g., a second element), it means that the element may be coupled with the other element directly (e.g., wired), wirelessly, or via at least a third element(s). Thus, “connected” as used herein covers both direct and indirect connections.

As used in connection with embodiments of the disclosure, the term “module” may include a unit implemented in hardware, software, or firmware, and may interchangeably be used with other terms, for example, “logic”, “logic block”, “part”, or “circuitry”. A module may be a single integral component, or a minimum unit or part thereof, adapted to perform one or more functions. For example, according to an embodiment, the module may be implemented in a form of an application-specific integrated circuit (ASIC). Thus, each “module” herein may comprise circuitry.

Embodiments as set forth herein may be implemented as software (e.g., the program 240) including one or more instructions that are stored in a storage medium (e.g., an internal memory 236 or an external memory 238) that is readable by a machine (e.g., the electronic device 201). For example, a processor (e.g., the processor 220) of the machine (e.g., the electronic device 201) may invoke at least one of the one or more instructions stored in the storage medium, and execute it. This allows the machine to be operated to perform at least one function according to the at least one instruction invoked. The one or more instructions may include code generated by a compiler or code executable by an interpreter. The machine-readable storage medium may be provided in the form of a non-transitory storage medium. Here, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

According to an embodiment, a method according to various embodiments of the disclosure may be included and provided in a computer program product. The computer program product may be traded as a product between a seller and a buyer. The computer program product may be distributed in the form of a machine-readable storage medium (e.g., compact disc read-only memory (CD-ROM)), or be distributed (e.g., downloaded or uploaded) online via an application store (e.g., PlayStore™), or between two user devices (e.g., smartphones) directly. If distributed online, at least a portion of the computer program product may be temporarily generated or at least temporarily stored in the machine-readable storage medium, such as memory of the manufacturer's server, a server of the application store, or a relay server.

According to an embodiment, each component (e.g., a module or a program) of the above-described components may include a single entity or multiple entities, and some of the multiple entities may be separately disposed in different components. According to an embodiment, one or more of the above-described components may be omitted, or one or more other components may be added. Alternatively or additionally, a plurality of components (e.g., modules or programs) may be integrated into a single component. In such a case, according to embodiments, the integrated component may still perform one or more functions of each of the plurality of components in the same or similar manner as they are performed by a corresponding one of the plurality of components before the integration. According to an embodiment, operations performed by the module, the program, or another component may be carried out sequentially, in parallel, repeatedly, or heuristically, or one or more of the operations may be executed in a different order or omitted, or one or more other operations may be added.

FIGS. 3A, 3B, 3C, and 3D are diagrams illustrating a wearable device, according to an embodiment.

Referring to FIGS. 3A, 3B, 3C, and 3D, a wearable device 300 (e.g., the wearable device 120 of FIG. 1) may be worn by a user to assist a gait of the user. For example, the wearable device 300 may be a device for assisting a gait of the user. Further, the wearable device 300 may be a workout device that provides a workout function by assisting a gait of the user and providing the user with a resistance force. For example, the resistance force provided to the user may be a force actively applied to the user, such as a force output by a device such as a motor. Alternatively, the resistance force may not be a force actively applied to the user but may be a force that impedes a motion of the user, such as a frictional force. The resistance force may also be referred to as an exercise load.

Although FIGS. 3A, 3B, 3C, and 3D illustrate a hip-type wearable device 300, the type of the wearable device is not limited thereto. The wearable device may be a type that supports the entire lower limbs or a type that supports a portion of the lower limbs. In addition, the wearable device may be one of a type that supports a portion of the lower limbs, a type that supports up to the knees, a type that supports up to the ankles, and a type that supports the entire body.

The embodiments described with reference to FIGS. 3A, 3B, 3C, and 3D may apply to a hip-type wearable device, but are not limited thereto, and may all apply to various types of wearable devices.

According to one aspect, the wearable device 300 may include a driver 310, a sensor unit 320, an inertial measurement unit (IMU) 330, a controller 340, a battery 350, and a communication module 352. For example, the IMU 330 and the controller 340 may be disposed in a main frame of the wearable device 300. Alternatively, the IMU 330 and the controller 340 may be included in a housing (not shown) that is formed in (or attached to) the outside of the main frame of the wearable device 300.

The driver 310 may include a motor 314 and a motor driver circuit 312 for driving the motor 314. The sensor unit 320 may include at least one sensor 321. The controller 340 may include a processor 342, a memory 344, and an input interface 346. Although one sensor 321, one motor driver circuit 312, and one motor 314 are shown in FIG. 3C, this is merely an example. As in another example shown in FIG. 3D, a wearable device 300-1 may include a plurality of sensors 321 and 321-1, a plurality of motor driver circuits 312 and 312-1, and a plurality of motors 314 and 314-1. Also, according to the implementation, the wearable device 300 may include a plurality of processors. The number of motor driver circuits, the number of motors, or the number of processors may vary depending on a body part on which the wearable device 300 is worn.

The following description of the sensor 321, the motor driver circuit 312, and the motor 314 may also apply to the sensor 321-1, the motor driver circuit 312-1, and the motor 314-1 illustrated in FIG. 3D.

The driver 310 may drive a hip joint of a user. For example, the driver 310 may be positioned on the right hip portion and/or the left hip portion of the user. The driver 310 may be additionally positioned on the knee portions and the ankle portions of the user. The driver 310 may include the motor 314 for generating a rotational torque and the motor driver circuit 312 for driving the motor 314.

The sensor unit 320 may measure the angles of the hip joints of the user during a gait. Information on the angles of the hip joints sensed by the sensor unit 320 may include the angle of the right hip joint, the angle of the left hip joint, the difference between the angles of both hip joints, and the hip joint motion direction. For example, the sensor 321 may be positioned in the driver 310. According to the position of the sensor 321, the sensor unit 320 may additionally measure the angles of the knees and the angles of the ankles of the user. For example, the sensor 321 may be an encoder. For example, the sensor 321 may be a hall sensor. The information on the angles of the joints measured by the sensor unit 320 may be transmitted to the controller 340.

According to one aspect, the sensor unit 320 may include a potentiometer. The potentiometer may sense an R-axis joint angle, an L-axis joint angle, an R-axis joint angular velocity, and an L-axis joint angular velocity according to a walking motion of the user. In this example, the R and L axes may be reference axes for the right leg and the left leg of the user, respectively. For example, the R/L axis may be set to be vertical to the ground and set such that a front side of a body of a person has a negative value and a rear side of the body has a positive value.

The IMU 330 may measure acceleration information and pose information during a gait. For example, the IMU 330 may sense X-axis, Y-axis, and Z-axis accelerations and X-axis, Y-axis, and Z-axis angular velocities according to the gait motion of the user. The acceleration information and pose information measured by the IMU 330 may be transmitted to the controller 340.

In addition to the sensor unit 320 and the IMU 330 described above, the wearable device 300 may include another sensor (e.g., an electromyogram (EMG) sensor) configured to sense a change in a quantity of motion of the user or a change in a biosignal according to a gait motion.

The controller 340 may control an overall operation of the wearable device 300. For example, the controller 340 may receive the information sensed by each of the sensor unit 320 and the IMU 330. The information sensed by the IMU 330 may include acceleration information and pose information, and the information sensed by the sensor unit 320 may include the angle of the right hip joint, the angle of the left hip joint, the difference between the angles of the two hip joints, and the hip joint motion direction.

According to an example embodiment, the controller 340 may also calculate the difference between the angles of both hip joints based on the angle of the right hip joint and the angle of the left hip joint. The controller 340 may generate a signal for controlling the driver 310 based on the sensed information. For example, the generated signal may be an assistance force for assisting a gait of the user. Alternatively, the generated signal may be a resistance force for impeding a gait of the user. The resistance force may be provided to the user to assist the user in doing an exercise.

In an example, the processor 342 of the controller 340 may control the driver 310 to provide the user with a resistance force.

For example, the driver 310 may provide the user with a resistance force by applying an active force to the user through the motor 314. The driver 310 may provide the resistance force to the user by outputting a torque in a direction that hinders a motion of the user.

Alternatively, the driver 310 may provide the user with a resistance force using the back-drivability of the motor 314, without applying an active force to the user. The back-drivability of the motor may be responsiveness of the rotation axis of the motor to an external force. When the back-drivability of the motor increases, the motor may more readily respond to an external force acting on the rotation axis of the motor (that is, the rotation axis of the motor may more readily rotate). For example, even when the same external force is applied to the rotation axis of the motor, a degree of rotation of the rotation axis of the motor may change according to a degree of the back-drivability.

According to an example embodiment, the processor 342 of the controller 340 may control the driver 310 such that the driver 310 may output a torque (or an assistance torque) for assisting a gait of the user. For example, in the hip-type wearable device 300, the driver 310 may be disposed on each of the left hip portion and the right hip portion, and the controller 340 may output a control signal for controlling the driver 310 to generate a torque.

The driver 310 may generate a torque based on the control signal output by the controller 340. A torque value for generating the torque may be externally set or be set by the controller 340. For example, to indicate a magnitude of the torque value, the controller 340 may use a magnitude of a current for the signal transmitted to the driver 310. That is, as the magnitude of the current received by the driver 310 increases, the torque value may increase. As another example, the processor 342 of the controller 340 may transmit the control signal to the motor driver circuit 312 of the driver 310, and the motor driver circuit 312 may generate a current corresponding to the control signal to control the motor 314.

The battery 350 may supply power to the components of the wearable device 300. The wearable device 300 may further include a circuit (e.g., a power management integrated circuit (PMIC)) configured to convert the power of the battery 350 according to an operating voltage of the components of the wearable device 300 and provide the same to the components of the wearable device 300. In addition, the battery 350 may or may not supply power to the motor 314 based on the operation mode of the wearable device 300.

The communication module 352 may support the establishment of a direct (or wired) communication channel or a wireless communication channel between the wearable device 300 and an external electronic device, and support the communication through the established communication channel. The communication module 352 may include one or more communication processors configured to support direct (e.g., wired) communication or wireless communication. According to an embodiment, the communication module 352 may include a wireless communication module (e.g., a cellular communication module, a short-range wireless communication module, or a global navigation satellite system (GNSS) communication module) or a wired communication module (e.g., a local area network (LAN) communication module, or a power line communication (PLC) module). A corresponding one of these communication modules may communicate with the external electronic device via a first network (e.g., a short-range communication network such as Bluetooth™, wireless-fidelity (Wi-Fi) direct, or infrared data association (IrDA)) or a second network (e.g., a legacy cellular network, a 5G network, a next-generation communication network, the Internet, or a computer network). These various types of communication modules may be implemented as a single component (e.g., a single chip), or may be implemented as multi components (e.g., multi chips) separate from each other.

FIG. 4 is a diagram illustrating a wearable device communicating with an electronic device, according to an embodiment.

Referring to FIG. 4, the wearable device 300 may communicate with the electronic device 201. For example, the electronic device 201 may be an electronic device of a user of the wearable device 300. According to an embodiment, the wearable device 300 and the electronic device 201 may be connected using a short-range wireless communication method.

The electronic device 201 may display a user interface (UI) for controlling an operation of the wearable device 300 on a display 201-1. The UI may include, for example, at least one soft key through which the user may control the wearable device 300.

The user may input a command for controlling the operation of the wearable device 300 through the UI on the display 201-1 of the electronic device 201, and the electronic device 201 may generate a control instruction corresponding to the command and transmit the generated control instruction to the wearable device 300. The wearable device 300 may operate according to the received control instruction, and transmit a control result to the electronic device 201. The electronic device 201 may display a control completion message on the display 201-1 of the electronic device 201.

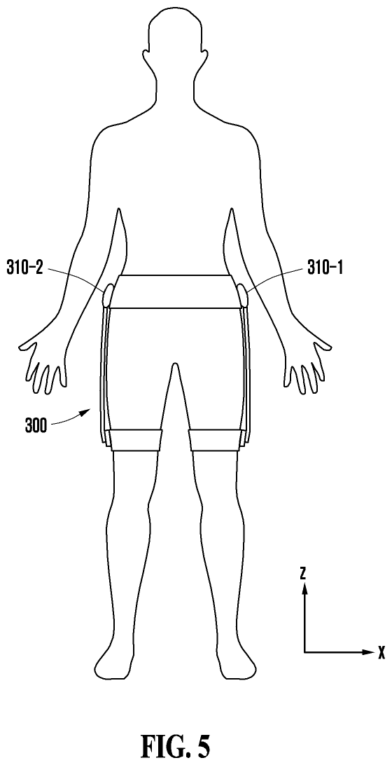

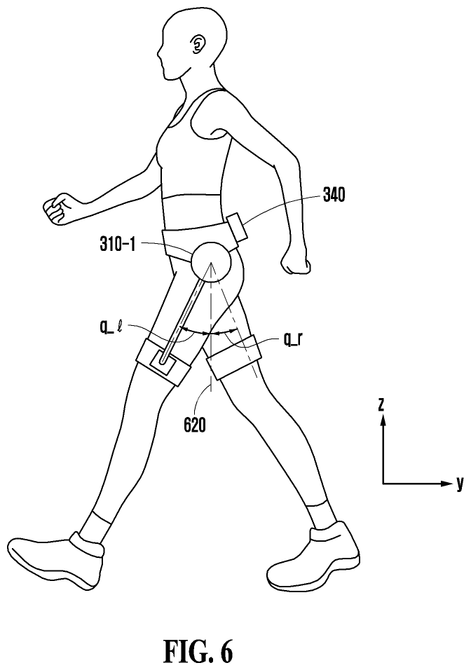

FIGS. 5 and 6 are diagrams illustrating a torque output method of a wearable device, according to an embodiment.

Referring to FIGS. 5 and 6, drivers 310-1 and 310-2 of the wearable device 300 of FIG. 3 may be disposed near the hip joints of a user, and the controller 340 of the wearable device 300 may be disposed near the lower back of the user. The positions of the drivers 310-1 and 310-2 and the controller 340 are not limited to the example positions illustrated in FIGS. 5 and 6.

The wearable device 300 may measure (or sense) a left hip joint angle q_l and a right hip joint angle q_r of the user. As an example, the wearable device 300 may measure the left hip joint angle q_l of the user through a left encoder and measure the right hip joint angle q_r of the user through a right encoder. As illustrated in FIG. 6, the left hip angle q_l may be negative because the left leg of the user is before a reference line 620, and the right hip angle q_r may be positive because the right leg of the user is behind the reference line 620. According to implementation, the right hip joint angle q_r may be negative when the right leg is before the reference line 620, and the left hip joint angle q_l may be positive when the left leg is behind the reference line 620. According to an embodiment, the wearable device 300 may obtain a first angle (e.g., q_r) and a second angle (e.g., q_l) by filtering a first raw angle (e.g., q_r_raw) of a first joint (e.g., the right hip joint) and a second raw angle (e.g., q_l_raw) of a second joint (e.g., the left hip joint) measured by the sensor unit 320. For example, the wearable device 300 may filter the first raw angle and the second raw angle based on a first previous angle and a second previous angle measured with respect to a previous time.

According to an embodiment, the wearable device 300 may determine a torque value τ(t) based on the left hip joint angle q_l, the right hip joint angle q_r, an offset angle c, a sensitivity α, a gain κ, and a delay Δt, and control the motor driver circuit 312 of the wearable device 300 to output the determined torque value τ(t). The force provided to the user by the torque value τ(t) may be referred to herein as force feedback. As an example, the wearable device 300 may determine the torque value τ(t) based on Equation 1 below.

$$y = sin ( q\_r ) ( q\_l ) \qquad [ Equation 1 ]$$

$$τ ( t ) = κ y ( t - Δ t )$$

In Equation 1, y denotes a state factor, q_r denotes the right hip joint angle, and q_l denotes the left hip joint angle. According to Equation 1, the state factor y may be associated with the distance between the two legs. For example, y being “0” may indicate a state (e.g., a crossing state) in which the distance between the legs is “0”, and the absolute value of y being maximum or high may indicate a state (e.g., a landing state) in which the angle between the legs is maximum or high. According to an embodiment, when q_r and q_l are measured at a time t, the state factor may be represented as y(t).

The gain κ is a parameter indicating the magnitude and direction of an output torque. As the magnitude of the gain κ increases, a greater torque may be output. If the gain κ is negative, a torque acting as a resistance force may be output to the user, and if the gain κ is positive, a torque acting as an assistance force may be output to the user. The delay Δt is a parameter associated with a torque output timing. A value of the gain k and a value of the delay Δt may be preset and may be adjusted by the user or the wearable device 300. A model configured to output a torque that is applied as an assistance force to the user based on Equation 1 and the parameters, such as the gain κ and the delay Δt may be a torque output model (or a torque output algorithm). The wearable device 300 may determine the magnitude and delay of a torque to be output by inputting the values of input parameters received through sensors into the torque output model.

According to an example embodiment, the wearable device 300 may determine a first torque value through Equation 2 below by applying a first gain value and a first delay value to a first state factor y(t), wherein the first gain value and the first delay value may be parameter values determined with respect to the state factor y(t).

$${τ}_{l} ( t ) = κ y ( t - Δ t ) \qquad [ Equation 2 ]$$

$${τ}_{r} ( t ) = - κ y ( t - Δ t )$$

The calculated first torque value may include a value for the first joint and a value for the second joint since it should be applied to the two legs. For example, τl(t) may be a value related to a left hip joint, which is the second joint and τr(t) may be a value related to a right hip joint, which is the first joint. τl(t) and τr(t) may have the same magnitude but the direction of the torque may be opposite to each other. The wearable device 300 may control the motor driver circuit 312 of the wearable device 300 to output a torque corresponding to the first torque value.

According to an embodiment, when the user performs an asymmetrical gait with the left leg and the right leg, the wearable device 300 may provide asymmetrical torques respectively to both legs of the user to assist the asymmetric gait. For example, a stronger assistance force may be provided to a leg with a shorter stride width or a slower swing speed. Hereinafter, a leg with a small stride width or a slow swing speed will be referred to as an affected leg or a target leg.

In general, an affected leg may have a shorter swing time or a smaller stride width than an unaffected leg. According to an embodiment, a method of adjusting the timing of a torque acting on an affected leg to assist a gait of a user may be considered. For example, an offset angle may be added to an actual joint angle of an affected leg to increase an output time of a torque to assist with a swing motion of the affected leg. As the offset angle is added to the actual joint angle of the affected leg, the value of an input parameter that is input into the torque output model mounted on (or applied to) the wearable device 300 may be adjusted. For example, values of q_r and q_l may be adjusted through Equation 3 below. cᵣ may denote an offset angle for the right hip joint and cₗ may denote an offset angle for the left hip joint.

$$q {\_}_{r} ( t ) ← q {\_}_{r} ( t ) + {c}_{r} \qquad [ Equation 3 ]$$

$$q {\_}_{l} ( t ) ← q {\_}_{l} ( t ) + {c}_{l}$$

According to an embodiment, the wearable device 300 may filter the state factor to reduce the discomfort the user may experience due to irregular torque outputs. For example, the wearable device 300 may determine an initial state factor y_{raw}(t) of a current time t based on the first angle of the first joint and the second angle of the second joint, and determine the first state factor y(t) based on a previous state factor y^{prv} determined with respect to a previous time t−1 and the initial state factor y_{raw}(t). The current time t may be a time at which t-th data (or sample) is processed, and the previous time t−1 may be a time at which t−1-th data is processed. For example, the difference between the current time t and the previous time t−1 may be an operation interval of a processor for generating or processing the corresponding items of data. The sensitivity α may be the value of a parameter indicating a sensitivity. For example, the sensitivity value may be continuously adjusted during a test gait. However, the sensitivity value may be preset to a predetermined value to reduce the computational complexity.

In the embodiment described above, a method of determining the values of the control parameters by the wearable device 300 has been described. However, the values of the control parameters may be determined by an electronic device (e.g., the electronic device 110 of FIG. 1 or the electronic device 201 of FIG. 2) instead of the wearable device 300. For example, the electronic device may receive sensing data from the wearable device 300, determine values of control parameters based on the sensing data, and control an operation of the wearable device 300 based on the determined values of the control parameters.

FIG. 7 is a flowchart of a method of controlling an output of a reference image based on first sensing information on a first motion of a user, according to an embodiment.

The following operations 710 to 740 may be performed to control an output of the reference image. Operations 710 to 750 may be performed by an electronic device (e.g., the electronic device 110 of FIG. 1 or the electronic device 201 of FIG. 2).

In operation 710, the electronic device may obtain first sensing information on a first motion of a user wearing a wearable device (e.g., the wearable device 120 of FIG. 1 or the wearable device 300 of FIG. 3). The electronic device may continuously receive the sensing information from the wearable device while the user performs an exercise. For example, the first sensing information may be information measured by at least one sensor (e.g., the IMU 330 or the sensor unit 320 of FIG. 3A) of the wearable device at a first time.

According to an embodiment, the first time may be a time point at which a motion of an exercise mode performed by the user is repeated. For example, when the exercise mode is a lunge mode, a time point at which an already performed motion of a plurality of classification motions (e.g., STEP 1 and STEP 2) forming the lunge is performed again may be determined to be the first time.

According to an embodiment, the first time may be a time point at which a motion of an exercise mode of an output reference image is repeated. For example, when the exercise mode is a lunge mode, a time point at which an already performed motion of a plurality of classification motions (e.g., STEP 1 and STEP 2) forming the lunge is performed again may be determined to be the first time.

In operation 720, the electronic device may determine a first target index for a first motion based on the first sensing information.

The target index may be data for structuring a motion of the user by numbers to be recognized by the electronic device. Typically, angles of various joints (e.g., left/right wrist joints, left/right elbow joints, left/right shoulder joints, left/right hip joints, left/right knee joints, and left/right ankle joints) of the user may vary while the user performs an exercise, but for a repetitive motion, a pattern in which the joints of the user change may be repetitive. For example, when the user performs a squat, an angle of the left hip joint and an angle of the right hip joint of the user may iteratively change in almost the same range. A plurality of indicators for a total range of the angle of the left hip joint and a total range of the angle of the right hip joint may be preset, wherein the total ranges may be obtained while a typical person performs a squat. When the squat is formed by a plurality of classification motions, indicators (or target indices) for angles of the right hip joint and the left hip joint may be determined with respect to the first motion of the user and a classification motion corresponding to the determined metrics (or the target indices) may be determined.

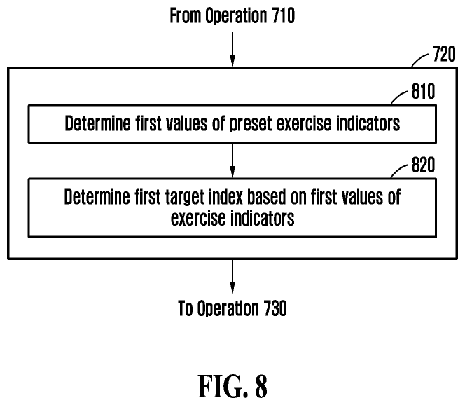

According to an embodiment, the electronic device may generate, as secondary information, a target index that is able to classify the motion of the user based on the sensing information, which is primary information. For example, the index may be a set of values of one or more exercise indicators determined based on one or more values of the obtained sensing information. A first target index determined with respect to the first motion of the user may indicate a current motion or a current posture of the user by integrating one or more exercise indicators. For example, each exercise indicator may be a value indicating a detailed posture (or an angle) of joints of the user. Different indices may indicate postures of different users. The postures indicated by the same indices may be postures that may be regarded as the same posture even if joint angles obtained with respect to the same joints are not exactly the identical to each other. The electronic device may determine a current classification motion among the plurality of classification motions for the exercise mode through the target index. Hereinafter, a method of determining a first target index is further described with reference to FIG. 8.

In operation 730, the electronic device may determine a first reference index of a reference image output at the first time.

According to an embodiment, the reference image may include reference index information that is preset to correspond to a motion shown on a screen. For example, when the reference image is an image for instructing lunge, the reference image may include sections (e.g., a first section and a second section) instructing a plurality of classification motions (e.g., STEP 1 and STEP 2) of the lunge. For example, the first section may be related to a reference index a indicating lunge STEP 1 and the second section may be related to a reference index b indicating lunge STEP 2. For example, when the first time is included in the first section, the electronic device may determine the reference index a to be the first reference index.

In operation 740, the electronic device may determine whether the first target index corresponds to the first reference index.

According to an embodiment, when the first target index is the same as the first reference index, the electronic device may determine that the first target index corresponds to the first reference index.

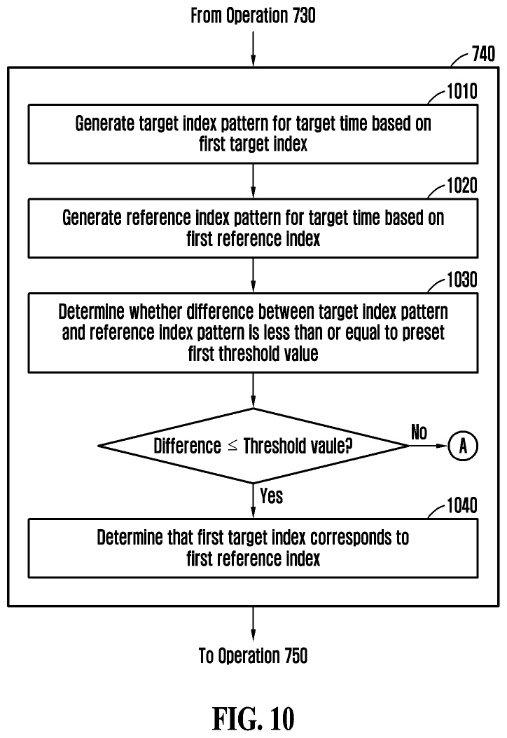

According to an embodiment, the electronic device may generate a target index pattern based on the first target index and may determine whether the first target index corresponds to the first reference index based on the target index pattern. The index pattern may indicate a change in indices over time. For example, the index pattern may indicate a flow of changes in a posture (or a classification motion) of the user over time. For example, in an embodiment in which the exercise mode performed by the user is different from the exercise mode of the output reference image but the first target index generated at the first time is incidentally the same as the first reference index when determining whether the first target index corresponds to the first reference index using the target index pattern, it may be determined that the first target index and the first reference index do not correspond to each other. A method of determining whether the first target index corresponds to the first reference index using the target index pattern is further described with reference to FIG. 10 below.

In operation 750, the electronic device may control an output of the reference image at a target time based on whether the first target index corresponds to the first reference index. The target time may be a time after the first time. For example, controlling the output of the reference image may be changing the output speed of the reference image. For example, controlling the output of the reference image may be changing an output section of the reference image. For example, controlling the output of the reference image may be changing a type of the reference image to another reference image (e.g., a second reference image).

FIG. 8 is a flowchart of a method of determining a first target index, according to an embodiment.

According to an embodiment, operation 720 described with reference to FIG. 7 may include the following operations 810 and 820. Operations 810 and 820 may be performed by an electronic device (e.g., the electronic device 110 of FIG. 1 or the electronic device 201 of FIG. 2).

In operation 810, the electronic device may determine first values of preset exercise indicators.

According to an embodiment, the exercise indicator may include a first exercise indicator indicating an angle difference between a left leg and a right leg. For example, the angle of the left leg may be an angle of the left hip joint (e.g., the left hip joint angle q_l of FIG. 6) and the angle of the right leg may be an angle of the right hip joint (e.g., the right hip joint angle q_r of FIG. 6).

According to an embodiment, the exercise indicator may include a second exercise indicator indicating an angle difference between the waist and the left leg. For example, the angle of the waist may be determined based on the information obtained by an IMU (e.g., the IMU 330 of FIG. 3A).

According to an embodiment, the exercise indicator may include a third exercise indicator indicating an angle difference between the waist and the right leg.

In operation 820, the electronic device may determine a first target index based on the first values of the exercise indicators.

According to an embodiment, the electronic device may index the first value of each exercise indicator based on an indexing range that is preset with respect to the exercise indicator. For example, a plurality of indexing sections may be set based on a maximum or high value and a minimum or low value indicated by the first exercise indicator. For example, when the maximum or high value of the exercise indicator indicating the angle difference between the left leg and the right leg is set to 100° and the minimum or low value thereof is set to −100°, the range between 100° and −100° may be divided into a plurality of indexing sections and an index value may be set to each indexing section. For example, when the plurality of indexing sections is divided into seven sections, the index values may include −3, −2, −1, 0, 1, 2, and 3.

According to an embodiment, when there are multiple exercise indicators, the index values may be determined for the exercise indicators, respectively. The index values (or an index set) of the exercise indicators at the first time may be referred to as the first target index.

According to an embodiment, the index values (or the index set) of the exercise indicators for the motion of the reference image that is output at the first time may be referred to as the first reference index.

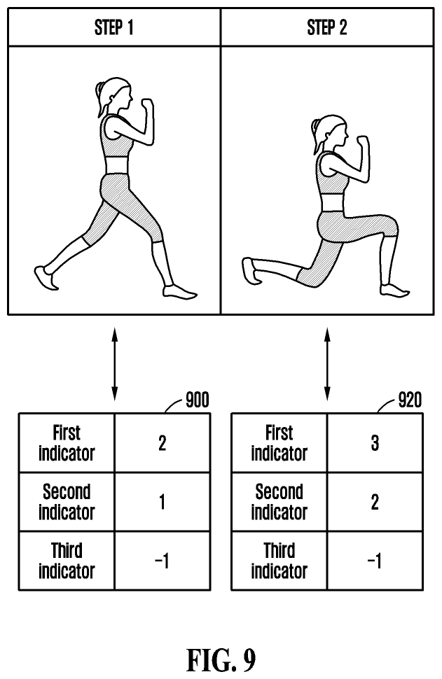

FIG. 9 illustrates reference indices for each of a plurality of classification motions forming an exercise mode, according to an embodiment.

According to an embodiment, the plurality of classification motions forming lunge may include lunge STEP 1 and lunge STEP 2. For example, the reference image for the lunge may be an image in which a motion of lunge STEP 1 and a motion of lunge STEP 2 are iteratively output.

According to an embodiment, the plurality of exercise indicators may include a first indicator indicating an angle difference between the left leg and the right leg, a second indicator indicating an angle difference between the waist and a first leg, and a third indicator indicating an angle difference between the waist and a second leg.

For example, in lunge STEP 1, the first indicator may indicate 2, the second indicator may indicate 1, and the third indicator may indicate −1. For lunge STEP 1, a first reference index 900 may be preset to {−2, 1, −1}.

For example, in lunge STEP 2, the first indicator may indicate 3, the second indicator may indicate 2, and the third indicator may indicate −1. For lunge STEP 2, a second reference index 920 may be preset to {3, 2, −1}.

According to an embodiment, the reference indices of the reference image of lunge are described with reference to FIG. 9 but the above description may apply identically or similarly to other exercise modes that are not described.

FIG. 10 is a flowchart of a method of determining whether a first target index corresponds to a first reference index, according to an embodiment.

According to an embodiment, operation 740 described with reference to FIG. 7 may include the following operations 1010 to 1040. Operations 1010 to 1040 may be performed by an electronic device (e.g., the electronic device 110 of FIG. 1 or the electronic device 201 of FIG. 2).

In operation 1010, the electronic device may generate a target index pattern for a target time based on a first target index. For example, the target time may be a previous time including a first time.

According to an embodiment, the target index pattern may include a plurality of target indices. For example, the target index pattern may include a first previous target index with respect to a first previous time and the first target index with respect to the first time.

For example, when the first time corresponds to lunge STEP 2, a first previous index may correspond to lunge STEP 1. For example, when the first previous index is {2, 1, −1} and the first target index is {3, 2, −1}, the target index pattern may be [{2, 1, −1}, {3, 2, −1}].

For example, when the first time corresponds to lunge STEP 1, the first previous index may correspond to lunge STEP 2. For example, when the first previous index is {3, 2, −1} and the first target index is {2, 1, −1}, the target index pattern may be [{3, 2, −1}, {2, 1, −1}].

In operation 1020, the electronic device may generate a reference index pattern for the target time based on a first reference index. For example, the target time may be the same as the target time described above with reference to operation 1010.

According to an embodiment, the target index pattern may include a plurality of reference indices. For example, the target index pattern may include a first previous reference index with respect to the first previous time and a first reference index with respect to the first time.

The description of the method of generating the reference index pattern may be replaced by the method of generating the target index pattern described with reference to operation 1010, and thereby a repeated description is omitted.

In operation 1030, the electronic device may determine whether a difference between the target index pattern and the reference index pattern is less than or equal to a preset first threshold value. For example, the difference may be calculated based on a difference between index values in the target index pattern and index values in the reference index pattern.

According to an embodiment, when the calculated difference between the target index pattern and the reference index pattern is less than or equal to the preset first threshold value, operation 1040 may be performed.

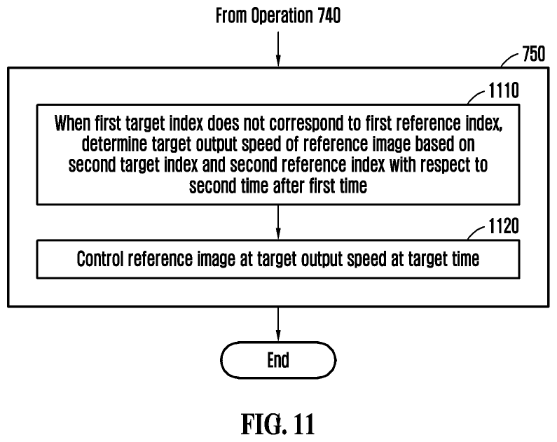

According to an embodiment, when the calculated difference between the target index pattern and the reference index pattern exceeds the preset first threshold value, it may be determined that the first target index does not correspond to the first reference index. When it is determined that the first target index does not correspond to the first reference index, operation 1110 of FIG. 11 below may be performed.

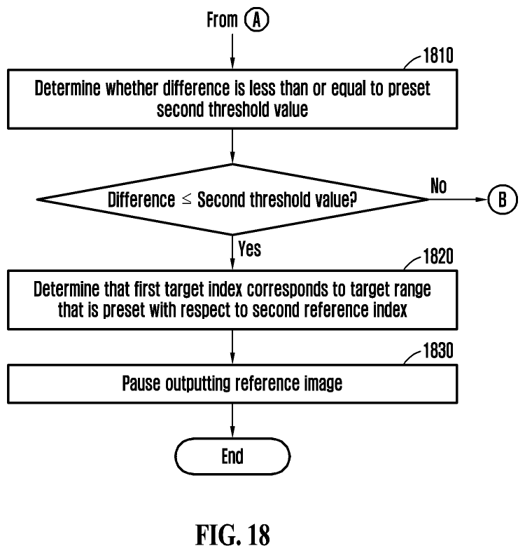

According to an embodiment, when the calculated difference between the target index pattern and the reference index pattern exceeds the preset first threshold value, operation A may be additionally performed. Operation A is further described with reference to FIG. 18 below.

In operation 1040, when the calculated difference between the target index pattern and the reference index pattern is less than or equal to the preset first threshold value, the electronic device may determine that the first target index corresponds to the first reference index.

According to an embodiment, when it is determined that the first target index corresponds to the first reference index in operation 1040 (or operation 740), in operation 750, the electronic device may output the reference image at the target time at the output speed of the reference image at the first time. For example, the output speed of the reference image at the first time and the output speed of the reference image at the target time may be the same.

FIG. 11 is a flowchart of a method of controlling an output of a reference image for a target sight when a first target index does not correspond to a first reference index, according to an embodiment.

According to an example embodiment, operation 750 described with reference to FIG. 7 may include the following operations 1110 and 1120. Operations 1110 and 1120 may be performed by an electronic device (e.g., the electronic device 110 of FIG. 1 or the electronic device 201 of FIG. 2).

According to an embodiment, operation 1110 may be performed when it is determined that the first target index does not correspond to the first reference index in operation 740 (or operation 1030).

In operation 1110, the electronic device may determine a target output speed of the reference image based on a second target index and a second reference index with respect to a second time after the first time.

For example, the second time may be the time at which a classification motion performed by the user changes. For example, the first time may correspond to lunge STEP 2 of the user and the second time may correspond to lunge STEP 1 of the user.

The descriptions of the first target index and the first reference index may apply similarly to the descriptions of the second target index and the second reference index, and thereby, a repeated description is omitted.

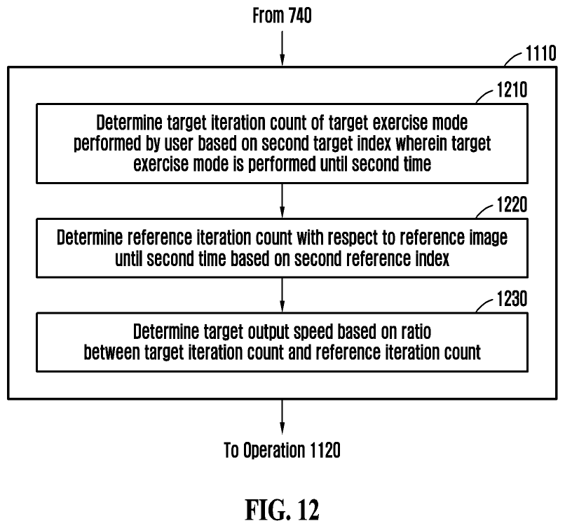

According to an embodiment, the electronic device may determine a target output speed of the reference image based on a motion iteration count of the motion of the user and a motion iteration count output on the reference image. The method of determining the target output speed of the reference image is further described with reference to FIG. 12 below.

In operation 1120, the electronic device may control the reference image at the target output speed at the target time.

According to an embodiment, the electronic device may synchronize the classification motion (e.g., lunge STEP 1) performed by the user at the target time with the classification motion (e.g., lunge STEP 1) of the output reference image and may control the reference image at the target output speed.

FIG. 12 is a flowchart of a method of determining a target output speed of a reference image when a first target index does not correspond to a first reference index, according to an embodiment.

According to an embodiment, operation 1110 described with reference to FIG. 11 may include the following operations 1210 to 1230. Operations 1210 to 1230 may be performed by an electronic device (e.g., the electronic device 110 of FIG. 1 or the electronic device 201 of FIG. 2).

In operation 1210, the electronic device may determine a target iteration count of a target exercise mode performed by the user based on the second target index, wherein the target exercise mode is performed until the second time.

According to an embodiment, the number of target indices generated until the second time may be determined to be the target iteration count. For example, the number of changes in classification motions may be determined to be the target iteration count based on the target indices generated until the second time.

In operation 1220, the electronic device may determine a reference iteration count with respect to the reference image until the second time based on the second reference index.

According to an embodiment, the number of reference indices generated until the second time may be determined to be the target iteration count. For example, the number of changes in the classification motions based on the reference indices generated until the second time may be determined to be the reference iteration count.

In operation 1230, the electronic device may determine the target output speed based on a ratio between the target iteration count and the reference iteration count. For example, when the target iteration count is 4 and the reference iteration count is 2, it may be determined that the speed of the motion of the user is twice as fast as the speed of the motion of the reference image. In the above case, the target output speed of the reference image may be determined to be twice the current output speed.

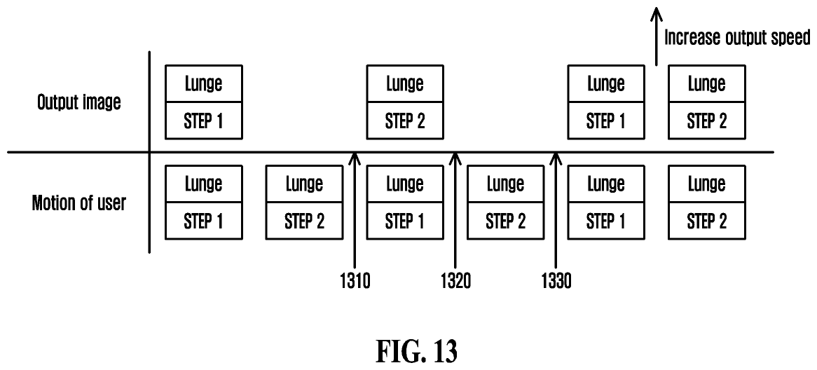

FIG. 13 illustrates a method of controlling an output of a reference image, according to an embodiment.

According to an embodiment, the user may perform lunge as an exercise mode and an output reference image may be an image for instructing the lunge. For example, the exercise speed of the user may be faster than the output speed of the reference image.

According to an embodiment, at a first time 1310 at which the user performs a plurality of classification motions (lunge STEP 1 and lunge STEP 2) once, an electronic device (e.g., the electronic device 110 of FIG. 1 or the electronic device 201 of FIG. 2) may determine that the first target index does not correspond to the first reference index. For example, at the first time 1310, the first target index may be {3, 2, −1} corresponding to lunge STEP 2 and the first reference index may be {2, 1, −1} corresponding to lunge STEP 1.

According to an embodiment, at a second time 1320, the electronic device may determine the target output speed of the reference image based on a second target index and a second reference index with respect to the second time 1320. For example, the second target index may be {2, 1, −1} corresponding to lunge STEP 1 and the second reference index may be {3, 2, −1} corresponding to lunge STEP 2. For example, the number of target indices until the second time 1320 may be three and the number of reference indices may be two. For example, since the progress of lunge STEP 2 of the reference image is not completed at the second time 1320, the number of reference indices may be adjusted from two to 1.5. According to the adjusted number of reference indices, since the number of target indices is twice the number of reference indices, the target output speed of the reference image may be determined to be twice.

According to an embodiment, at a third time 1330, the electronic device may synchronize a classification motion (e.g., lunge STEP 1) performed by the user with a classification motion (e.g., lunge STEP 1) of the output reference image and may control the target output speed (e.g., twice) of the reference image.

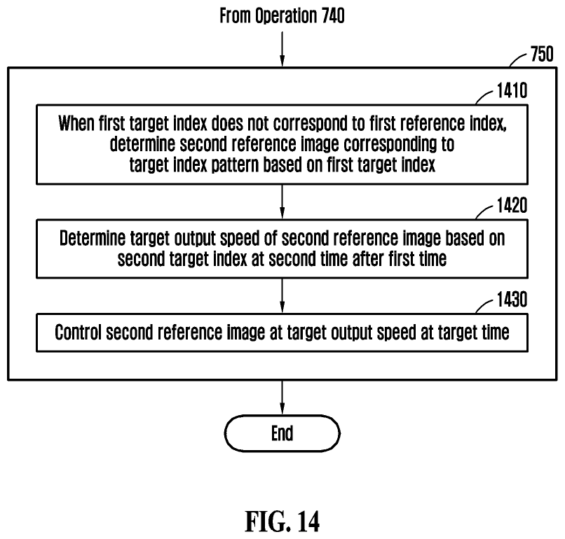

FIG. 14 is a flowchart of a method of controlling an output of a second reference image corresponding to a target index pattern of a first target index, according to an embodiment.

According to an embodiment, operation 750 described with reference to FIG. 7 may include the following operations 1410 to 1430. Operations 1410 to 1430 may be performed by an electronic device (e.g., the electronic device 110 of FIG. 1 or the electronic device 201 of FIG. 2).

According to an embodiment, operation 1410 may be performed when it is determined that the first target index does not correspond to the first reference index in operation 1030 described above with reference to FIG. 10.

In operation 1410, the electronic device may determine a second reference image corresponding to the target index pattern generated based on the first target index.

According to an embodiment, when the reference image with respect to lunge is output while the user performs a squat as an exercise mode, the first target index generated based thereon and the first reference index may not correspond to each other. For example, the electronic device may determine whether the reference image corresponds to the exercise mode performed by the user by comparing the target index pattern generated based on the first target index with the reference index pattern generated based on a first reference index. For example, when the reference image corresponds to the example mode performed by the user, the target index and the reference index at the same time may not be the same because the output speed is not synchronized, but the target index pattern and the index pattern of the reference image may correspond to each other. For example, when the reference image does not correspond to the exercise mode performed by the user, the target index pattern and the pattern of the reference image may not correspond to each other.

According to an embodiment, the electronic device may determine a second reference image pattern corresponding to the target index pattern from a plurality of reference index patterns for a plurality of reference images. A second reference image related to the determined second reference image pattern may be determined.

In operation 1420, the electronic device may determine the target output speed of the second reference image based on the second target index at the second time after the first time. For example, the electronic device may determine the target iteration count of the target exercise mode performed by the user based on the second target index, wherein the target exercise mode is performed until the second time.

According to an embodiment, the number of target indices generated until the second time may be determined to be the target iteration count. For example, the number of changes in classification motions may be determined to be the target iteration count based on the target indices generated until the second time.

According to an embodiment, the electronic device may determine the target output speed of the second reference image based on the target iteration count of the target exercise mode. For example, the target output speed of the second reference image may be determined to correspond to an iteration cycle of the classification motion of the user.

In operation 1430, the electronic device may control the second reference image at the target output speed at the target time.

According to an embodiment, the electronic device may synchronize a classification motion (e.g., squat STEP 1) performed by the user at the target time with a classification motion (e.g., squat STEP 1) of the output second reference image and may control the second reference image at the target output speed.

According to an embodiment, before performing operation 1430, the electronic device may ask the user whether to change from the reference image to the second reference image and when the user accepts to output the second reference image, the electronic device may perform operation 1430.

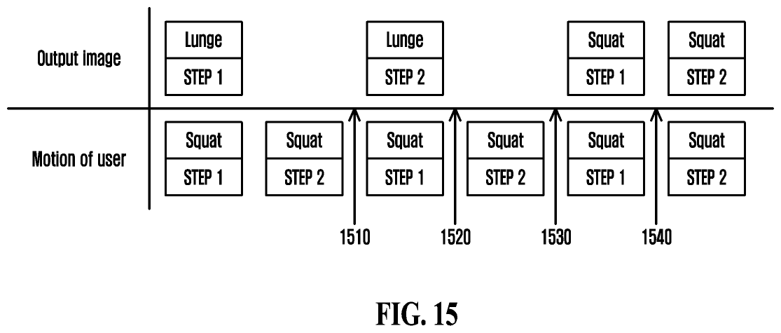

FIG. 15 is a method of controlling an output of a modified second reference image, according to an embodiment.

According to an embodiment, the user may perform a squat as an exercise mode and an output reference image may be an image for instructing lunge. For example, the exercise speed of the user may be faster than the output speed of the reference image.

According to an embodiment, at a first time 1510 at which the user performs a plurality of classification motions (squat STEP 1 and squat STEP 2) once, an electronic device (e.g., the electronic device 110 of FIG. 1 or the electronic device 201 of FIG. 2) may determine that the first target index does not correspond to the first reference index. For example, at the first time 1510, the first target index may be {0, 2, 2} corresponding to squat STEP 2 and the first reference index may be {2, 1, −1} corresponding to lunge STEP 1.

According to an embodiment, when the first target index and the first reference index do not correspond to each other, the electronic device may determine the second reference image based on the first target index.

According to an embodiment, at a second time 1520, the electronic device may determine the target output speed of the second reference image based on the second target index with respect to the second time 1520. For example, the electronic device may determine that an iteration cycle of the user is twice as fast as a default speed using the target index pattern generated based on the second target index.

According to an embodiment, at a third time 1530, the electronic device may inquire the user whether to change from the reference image to the second reference image. When the user accepts to output the second reference image, the second reference image may be output at the following time.

According to an embodiment, at a fourth time 1540, the electronic device may synchronize a classification motion (e.g., squat STEP 1) performed by the user with a classification motion (e.g., squat STEP 1) of the output second reference image and may control the second reference image at the target output speed (e.g., twice).

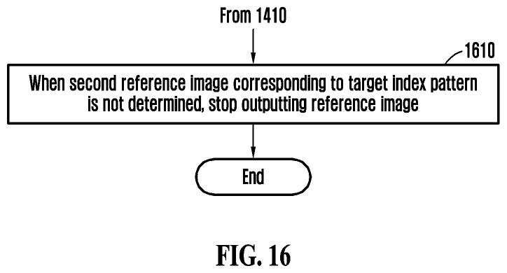

FIG. 16 is a flowchart of a method of stopping outputting a reference image, according to an embodiment.

According to an embodiment, after operation 1410 described above with reference to FIG. 14 is performed, the following operation 1610 may be performed. Operation 1610 may be performed by an electronic device (e.g., the electronic device 110 of FIG. 1 or the electronic device 201 of FIG. 2).

In operation 1610, when the second reference image corresponding to the target index pattern is not determined, the electronic device may stop outputting the reference image.

According to an embodiment, when none of the plurality of reference indices corresponds to the target index pattern, the electronic device may determine that the second reference image corresponding to the target index pattern is not determined.

According to an embodiment, the user may change the exercise mode to be performed from lunge to donkey kick. When the exercise mode is changed to donkey kick while the electronic device outputs a lunge image corresponding to lunge, the electronic device may determine the incongruity of the generated target index pattern and the reference index pattern. When the incongruity of the generated target index pattern and the reference index pattern is determined, the electronic device may output a guide image informing that an exercise mode that is different from the current exercise mode is detected.

According to an embodiment, when the reference index pattern related to a donkey kick is not stored in the electronic device, the electronic device may not determine a new exercise mode corresponding to the target index pattern. For example, when the new exercise mode corresponding to the target index pattern is not determined, the electronic device may stop outputting the reference image that is currently output.

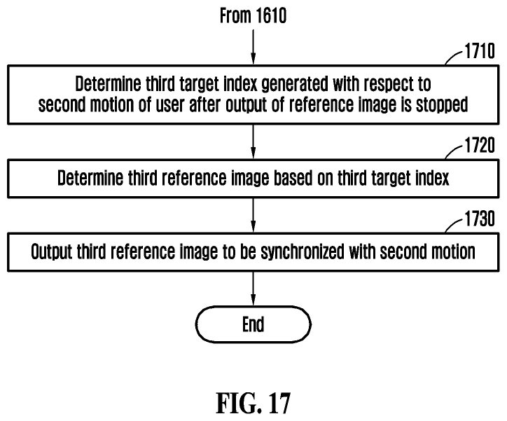

FIG. 17 is a flowchart of a method of outputting a third reference image based on a second motion of a user after outputting a reference image is stopped, according to an embodiment.

According to an embodiment, after operation 1610 described above with reference to FIG. 16 is performed, the following operations 1710 to 1730 may be performed. Operations 1710 to 1730 may be performed by an electronic device (e.g., the electronic device 110 of FIG. 1 or the electronic device 201 of FIG. 2).

In operation 1710, the electronic device may determine a third target index generated with respect to a second motion of the user after the output of the reference image is stopped.

According to an embodiment, the user may change the exercise mode to be performed from donkey kick to squat. The determined third target index may be a target index generated with respect to the motion of a squat.

In operation 1720, the electronic device may determine a third reference image based on the third target index.

According to an embodiment, the electronic device may determine a third reference index corresponding to the third index from a plurality of reference indices. The electronic device may determine the third reference image related to the third reference index.

According to an embodiment, the electronic device may generate the third target index pattern based on the third target index. The electronic device may determine the third reference index pattern corresponding to the third target index from a plurality of reference index patterns. The electronic device may determine the third reference image related to the third reference image index pattern.

In operation 1730, the electronic device may output the third reference image to be synchronized to a second motion.

According to an embodiment, the electronic device may determine the output speed of the third reference image based on the third target index (or the third target index pattern). Since the description of operations 1210 to 1230 described above with reference to FIG. 12 may similarly apply to the description of the method of determining the output speed of the third reference image, a repeated description is omitted.

According to an embodiment, while the user performs a first classification motion (e.g., squat STEP 1) of the second motion, the electronic device may synchronize an output time instant of the third reference image with the second motion to output a first classification motion (e.g., squat STEP 1) of the third reference image for a squat. The electronic device may output the third reference image at the determined output speed after the synchronization time point.

FIG. 18 is a flowchart of pausing outputting a reference image when a motion of a user is inaccurate, according to an embodiment.

According to an embodiment, after operation 1030 described above with reference to FIG. 10 is performed, the following operations 1810 to 1830 may be performed. For example, in operation 1030, when the calculated difference between the target index pattern and the reference index pattern exceeds the preset first threshold value, operation 1810 may be performed.

According to an embodiment, operation 740 described with reference to FIG. 7 may include operations 1810 and 1820 and operation 750 may include operation 1830.

Operations 1810 to 1830 may be performed by an electronic device (e.g., the electronic device 110 of FIG. 1 or the electronic device 201 of FIG. 2).

According to an embodiment, operations 1810 to 1830 may be performed to detect a case in which the user performs a specific exercise mode but a performed motion is inaccurate.

In operation 1810, the electronic device may determine whether the calculated difference between the target index pattern and the reference index pattern is less than or equal to a preset second threshold value. For example, the second threshold value may be a value greater than the first threshold value.

According to an embodiment, when the calculated difference between the target index pattern and the reference index pattern is less than or equal to the preset second threshold value, operation 1820 may be performed.

According to an embodiment, when the calculated difference between the target index pattern and the reference index pattern exceeds the preset second threshold value, it may be determined that the first target index does not correspond to the first reference index. For example, when the specific exercise mode performed by the user does not correspond to an exercise mode of the reference image, the calculated difference between the target index pattern and the reference index pattern may exceed the preset second threshold value. When it is determined that the first target index does not correspond to the first reference index, operation B may be additionally performed.

According to an embodiment, in operation B, the electronic device may stop outputting the reference image. According to an embodiment, after operation B is performed, operation 1710 described above with reference to FIG. 17 may be performed. In operation 1820, the electronic device may determine that the first target index corresponds to a target range that is preset with respect to the first reference index.

According to an embodiment, even though the user performs lunge as the exercise mode, a performed posture may be inaccurate. In the above case, the electronic device may determine that the first target index corresponds to the target range that is preset with respect to the first reference index.

In operation 1830, the electronic device may pause outputting the reference image.

According to an embodiment, when the output of the reference image is stopped and the exercise posture is accurate, the electronic device may output a guide image informing that the output of the reference image is resumed.

According to an embodiment, when the exercise-performing posture of the user corresponds to the exercise mode of the reference image, the electronic device may resume outputting the reference image.

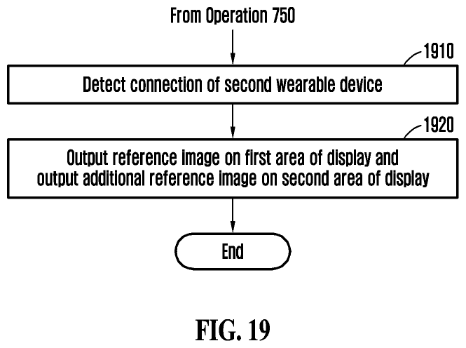

FIG. 19 is a flowchart of a method of outputting an additional reference image when a connection of a second wearable device is detected, according to an embodiment. According to an embodiment, after operation 750 described above with reference to FIG. 7 is performed, the following operations 1910 and 1920 may be performed. Operations 1910 and 1920 may be performed by an electronic device (e.g., the electronic device 110 of FIG. 1 or the electronic device 201 of FIG. 2).

In operation 1910, the electronic device may detect the connection of a second wearable device. For example, the second wearable device may be a wearable device worn by a second user.

According to an embodiment, while being connected to a wearable device (e.g., the wearable device 120 of FIG. 1 or the wearable device 300 of FIG. 3), the electronic device may be additionally connected to the second wearable device. For example, the second user may perform an exercise mode using the second wearable device. For example, the electronic device may receive, through the second wearable device, information about the exercise mode performed by the second user. For example, the electronic device may receive sensing information from the second wearable device and may determine information about the exercise mode performed by the second user based on the received sensing information. For exercise mode, the electronic device may determine an additional reference image based on the information about the exercise mode performed by the second user.

In operation 1920, the electronic device may output the reference image for the wearable device on a first area of a display and may output the additional reference image for the second wearable device on a second area of the display. For example, the display may be a display (e.g., a display of the display module 260 of FIG. 2) included in the electronic device. For example, the display may be a display of another electronic device (e.g., a television or a monitor) connected to the electronic device.

According to an embodiment, when the output speed of the reference image is adjusted (e.g., increases or decreases), the electronic may control the output speed of the additional reference image to correspond to the output speed of the reference image.

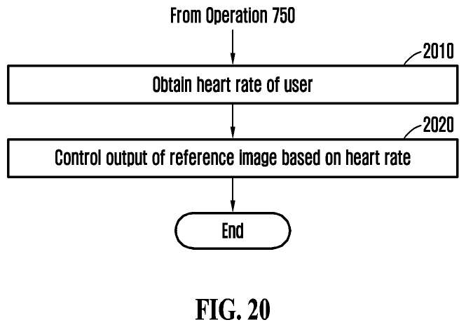

FIG. 20 is a flowchart of a method of controlling outputting a reference image based on a heart rate of a user, according to an embodiment.

According to an embodiment, after operation 750 described above with reference to FIG. 7 is performed, the following operations 2010 and 2020 may be performed. Operations 2010 and 2020 may be performed by an electronic device (e.g., the electronic device 110 of FIG. 1 or the electronic device 201 of FIG. 2).

In operation 2010, the electronic device may obtain a heart rate of the user. For example, the electronic device may receive the heart rate of the user from an accessory device (e.g., the smartwatch 132 of FIG. 1). For example, the electronic device may receive the heart rate of the user from a wearable device (e.g., the wearable device 120 of FIG. 1 or the wearable device 300 of FIG. 3).

In operation 2020, the electronic device may control an output of the reference image based on the heart rate.

According to an embodiment, the electronic device may compare a threshold heart rate that is preset with respect to the user or the exercise mode performed by the user with a received current heart rate of the user.

According to an embodiment, when the current heart rate of the user is lower than a lower threshold heart rate, the electronic device may increase the output speed of the reference image. When the output speed of the reference image increases, the user may increase the exercise-performing speed to follow the reference image at the increased output speed.

According to an embodiment, when the current heart rate of the user is higher than an upper threshold heart rate, the electronic device may decrease the output speed of the reference image. When the output speed of the reference image decreases, the user may decrease the exercise-performing speed to follow the reference image at the decreased output speed.

According to an embodiment, when the current heart rate of the user is higher than a maximum threshold heart rate, the electronic device may stop outputting the reference image. When the output of the reference image is stopped, the user may stop the excessive exercise currently being performed.

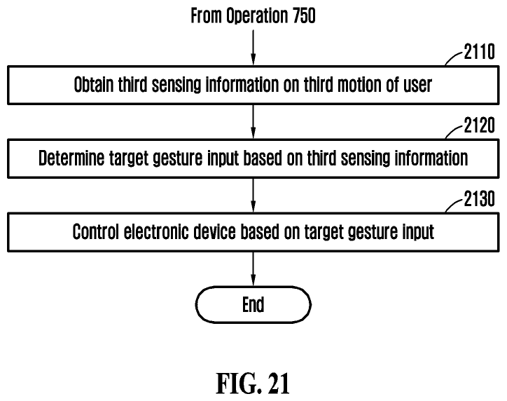

FIG. 21 is a flowchart of a method of controlling an electronic device based on a target gesture input, according to an embodiment.

According to an embodiment, after operation 750 described above with reference to FIG. 7 is performed, the following operations 2110 to 2130 may be performed. Operations 2110 to 2130 may be performed by an electronic device (e.g., the electronic device 110 of FIG. 1 or the electronic device 201 of FIG. 2).

According to an embodiment, operation 2110 may be performed while an output of the reference image is stopped as the user stops performing the exercise.

In operation 2110, the electronic device may obtain third sensing information about a third motion of the user. For example, the user wearing a wearable device (e.g., the wearable device 120 of FIG. 1 or the wearable device 300 of FIG. 3) may perform a body motion (e.g., the third motion) representing a gesture to generate a user input. For example, the gesture may include stretching the left leg to the side, stretching the right leg to the side, left knee-up, or right knee-up, and the embodiment of the gesture is not limited thereto. The wearable device may generate the third sensing information about the motion and may transmit the generated third sensing information to the electronic device.

In operation 2120, the electronic device may determine a target gesture input based on the third sensing information. For example, a target gesture index may be determined based on the third sensing information. For example, the target gesture index may be an index generated based on the plurality of exercise indicators described above with reference to FIG. 8.

According to an embodiment, the electronic device may determine the target gesture input corresponding to the target gesture index generated based on the third sensing information. For example, the electronic device may determine the target gesture input corresponding to the target gesture index from the plurality of target gesture inputs. For example, stretching the right leg to the side may be preset to a gesture input to “execute a background app”. For example, stretching the left leg to the side may be preset to a gesture input to “return to the exercise image”.

In operation 2130, the electronic device may control the electronic device based on the target gesture input. The electronic device may control the electronic device to perform the determined target gesture input. For example, when the target gesture input is “executing a background app”, one or more apps that are running in the background or are disabled may be displayed on a display. For example, when the target gesture input is “returning to the exercise image”, the output of the reference image may be resumed in the electronic device.

According to an example embodiment, the electronic device 110; 201 may include the communication module 290 configured to exchange data with an external device, and at least one processor 220 configured to control the electronic device, wherein the processor 220 is configured to perform operation 710 of obtaining first sensing information with respect to a first motion of a user wearing a wearable device 120, 300, wherein the first sensing information is measured at a first time, operation 720 of determining a first target index for the first motion based on the first sensing information, operation 730 of determining a first reference index of a reference image output at the first time, operation 740 of determining whether the first target index corresponds to the first reference index, and operation 750 of controlling an output of the reference image at a target time after the first time based on whether the first target index corresponds to the first reference index.

According to an embodiment, operation 720 of determining the first target index for the first motion based on the first sensing information may include operation 810 of determining first values of one or more preset exercise indicators, and operation 820 of determining the first target index based on the first values of the exercise indicators.

According to an embodiment, operation 740 of determining whether the first target index corresponds to the first reference index may include operation 1010 of generating a target index pattern with respect to a preset target time based on the first target index, operation 1020 of generating a reference index pattern with respect to the target time based on the first reference index, operation 1030 of determining whether a difference between the target index pattern and the reference index pattern is less than or equal to a preset first threshold value, and operation 1040 of determining that the first target index corresponds to the first reference index when the difference is less than or equal to the first threshold value.

According to an embodiment, operation 750 of controlling the output of the reference image at the target time after the first time based on whether the first target index corresponds to the first reference index may include, when the first target index corresponds to the first reference index, an operation of outputting the reference image at the target time at an output speed of the reference image at the first time.

According to an embodiment, operation 750 of controlling the output speed of the reference image at the target time after the first time may include, when the first target index does not correspond to the first reference index, operation 1110 of determining a target output speed of the reference image based on a second target index and a second reference index with respect to a second time after the first time, and operation 1120 of controlling the reference image at the target output speed at the target time.

According to an embodiment, operation 1110 of determining the target output speed of the reference image based on the second target index and the second reference index with respect to the second time may include operation 1210 of determining a target iteration count of a target exercise mode performed by the user based on the second target index, wherein the target exercise mode is performed until the second time, operation 1220 of determining a reference iteration count for the reference image until the second time based on the second reference index, and operation 1230 of determining the target output speed based on a ratio between the target iteration count and the reference iteration count.

According to an embodiment, operation 750 of controlling the output of the reference image at the target time after the first time based on whether the first target index corresponds to the first reference index may include, when the first target index does not correspond to the first reference index, operation 1410 of determining a second reference image corresponding to a target index pattern generated based on the first target index, operation 1420 of determining a target output speed of the second reference image based on a second target index with respect to the second time after the second time, and operation 1430 of controlling the second reference image at the target output speed at the target time.

According to an embodiment, operation 750 of controlling the output of the reference image at the target time after the first time based on whether the first target index corresponds to the first reference index may further include operation 1610 of stopping outputting the reference image when the second reference image corresponding to the target index pattern is not determined.

According to an embodiment, the processor 220 may be further configured to perform operation 1710 of determining a third target index generated with respect to a second motion of the user after the output of the reference image is stopped, operation 1720 of determining a third reference image based on the third target index, and operation 1730 of outputting the third reference image to synchronize with the second motion.

According to an embodiment, operation 740 of determining whether the first target index corresponds to the first reference index may further include, when the difference between the target index pattern and the reference index pattern exceeds the first threshold value, operation 1810 of determining whether the difference is less than or equal to a preset second threshold value, and when the difference is less than or equal to the second threshold value, operation 1820 of determining that the first target index corresponds to a target range that is preset with respect to the first reference index.

According to an embodiment, operation 750 of controlling the output of the reference image at the target time after the first time based on whether the first target index corresponds to the first reference index may further include operation 1830 of pausing the output of the reference image when it is determined that the first target index corresponds to the target range that is preset with respect to the first reference index.

According to an embodiment, the processor 220 may be further configured to perform operation 1920 of outputting the reference image on a first area of a display 260 and outputting an additional reference image on a second area of the display 260 for a second user wearing a second wearable device.

According to an embodiment, operation 1920 of outputting the additional reference information on the second area of the display for the second user may include an operation of controlling an output speed of the additional reference image based on the output speed of the reference image.

According to an embodiment, the processor 220 may be further configured to perform operation 2010 of obtaining a heart rate of the user, and operation 2020 of controlling the output of the reference image based on the heart rate.

According to an embodiment, operation 2020 of controlling the output of the reference image based on the heart rate may include an operation of increasing the output speed of the reference image when the heart rate is lower than a lower threshold heart rate that is preset with respect to the reference image.

According to an embodiment, operation 2020 of controlling the output of the reference image based on the heart rate may include an operation of decreasing the output speed of the reference image when the heart rate is higher than an upper threshold heart rate that is preset with respect to the reference image.

According to an embodiment, a method, performed by the electronic device 110, 201, of outputting a reference image may include operation 710 of obtaining first sensing information with respect to a first motion of a user wearing a wearable device 120, 300, wherein the first sensing information is measured at a first time, operation 720 of determining a first target index for the first motion based on the first sensing information, operation 730 of determining a first reference index of a reference image output at the first time, operation 740 of determining whether the first target index corresponds to the first reference index, and operation 750 of controlling an output of the reference image at a target time after the first time based on whether the first target index corresponds to the first reference index.

According to an embodiment, operation 720 of determining the first target index for the first motion based on the first sensing information may include operation 810 of determining first values of one or more preset exercise indicators, and operation 820 of determining the first target index based on the first values of the exercise indicators.

According to an embodiment, operation 740 of determining whether the first target index corresponds to the first reference index may include operation 1010 of generating a target index pattern with respect to a preset target time based on the first target index, operation 1020 of generating a reference index pattern with respect to the target time based on the first reference index, operation 1030 of determining whether a difference between the target index pattern and the reference index pattern is less than or equal to a preset first threshold value, and operation 1040 of determining that the first target index corresponds to the first reference index when the difference is less than or equal to the first threshold value.

According to an embodiment, operation 750 of controlling the output of the reference image at the target time after the first time based on whether the first target index corresponds to the first reference index may include, when the first target index does not correspond to the first reference index, operation 1410 of determining a second reference image corresponding to a target index pattern generated based on the first target index, operation 1420 of determining a target output speed of the second reference image based on a second target index with respect to the second time after the second time, and operation 1430 of controlling the second reference image at the target output speed at the target time. “Based on” as used herein covers based at least on.

According to an embodiment, operation 750 of controlling the output of the reference image at the target time after the first time based on whether the first target index corresponds to the first reference index may further include operation 1610 of stopping outputting the reference image when the second reference image corresponding to the target index pattern is not determined.

The embodiments described herein may be implemented using a hardware component, a software component and/or a combination thereof. A processing device may be implemented using one or more general-purpose or special-purpose computers, such as, for example, a processor, a controller and an arithmetic logic unit (ALU), a DSP, a microcomputer, a field-programmable gate array (FPGA), a programmable logic unit (PLU), a microprocessor or any other device capable of responding to and executing instructions in a defined manner. The processing device may run an operating system (OS) and one or more software applications that run on the OS. The processing device also may access, store, manipulate, process, and create data in response to execution of the software. For purpose of simplicity, the description of a processing device is used as singular; however, one skilled in the art will appreciate that a processing device may include multiple processing elements and multiple types of processing elements. For example, the processing device may include a plurality of processors, or a single processor and a single controller. In addition, different processing configurations are possible, such as parallel processors.

The software may include a computer program, a piece of code, an instruction, or some combination thereof, to independently or uniformly instruct or configure the processing device to operate as desired. Software and data may be embodied permanently or temporarily in any type of machine, component, physical or pseudo equipment, computer storage medium or device, or in a propagated signal wave capable of providing instructions or data to or being interpreted by the processing device. The software also may be distributed over network-coupled computer systems so that the software is stored and executed in a distributed fashion. The software and data may be stored by one or more non-transitory computer-readable recording mediums.

The methods according to the above-described embodiments may be recorded in non-transitory computer-readable media including program instructions to implement various operations of the above-described embodiments. The media may also include, alone or in combination with the program instructions, data files, data structures, and the like. The program instructions recorded on the media may be those specially designed and constructed for the purposes of embodiments, or they may be of the kind well-known and available to those having skill in the computer software arts. Examples of non-transitory computer-readable media include magnetic media such as hard disks, floppy disks, and magnetic tape; optical media such as CD-ROM discs, DVDs, and/or Blue-ray discs; magneto-optical media such as optical discs; and hardware devices that are specially configured to store and perform program instructions, such as read-only memory (ROM), random access memory (RAM), flash memory (e.g., USB flash drives, memory cards, memory sticks, etc.), and the like. Examples of program instructions include both machine code, such as produced by a compiler, and files containing higher-level code that may be executed by the computer using an interpreter.

The above-described devices may be configured to act as one or more software modules in order to perform the operations of the above-described examples, or vice versa.

As described above, although the embodiments have been described with reference to the limited drawings, a person skilled in the art may apply various technical modifications and variations based thereon. For example, suitable results may be achieved if the described techniques are performed in a different order and/or if components in a described system, architecture, device, or circuit are combined in a different manner and/or replaced or supplemented by other components or their equivalents. While the disclosure has been illustrated and described with reference to various embodiments, it will be understood that the various embodiments are intended to be illustrative, not limiting. It will further be understood by those skilled in the art that various changes in form and detail may be made without departing from the true spirit and full scope of the disclosure, including the appended claims and their equivalents. It will also be understood that any of the embodiment(s) described herein may be used in conjunction with any other embodiment(s) described herein. Accordingly, other implementations are within the scope of the following claims.

1. An electronic device comprising:
a communication module, comprising communication circuitry, configured to exchange data with an external device; and
at least one processor, comprising processing circuitry, individually and/or collectively configured to control the electronic device to perform:
an operation obtaining first sensing information with respect to a first motion of a user of a wearable device, wherein the first sensing information is to be measured at a first time;
an operation determining a first target index for the first motion based on the first sensing information;
an operation determining a first reference index of a reference image output at the first time;
an operation determining whether the first target index corresponds to the first reference index; and
an operation controlling an output of the reference image at a target time after the first time based on whether the first target index corresponds to the first reference index.

2. The electronic device of claim 1,
wherein the operation determining the first target index for the first motion based on the first sensing information comprises:
determining first values of one or more preset exercise indicators; and
determining the first target index based on the first values of the exercise indicators.

3. The electronic device of claim 1,
wherein the operation determining whether the first target index corresponds to the first reference index comprises:
generating a target index pattern with respect to a preset target time based on the first target index;
generating a reference index pattern with respect to the target time based on the first reference index;
determining whether a difference between the target index pattern and the reference index pattern is less than or equal to a preset first threshold value; and
determining that the first target index corresponds to the first reference index when the difference is less than or equal to the first threshold value.

4. The electronic device of claim 3,
wherein the operation controlling the output of the reference image at the target time after the first time based on whether the first target index corresponds to the first reference index comprises:
when the first target index corresponds to the first reference index, outputting the reference image at the target time at an output speed of the reference image at the first time.

5. The electronic device of claim 3,
wherein the operation controlling the output of the reference image at the target time after the first time comprises:
when the first target index does not correspond to the first reference index, determining a target output speed of the reference image based on a second target index and a second reference index with respect to a second time after the first time; and
controlling the reference image at the target output speed at the target time.

6. The electronic device of claim 5,
wherein determining the target output speed of the reference image based on the second target index and the second reference index with respect to the second time comprises:
determining a target iteration count of a target exercise mode performed by the user based on the second target index, wherein the target exercise mode is to be performed until the second time;
determining a reference iteration count for the reference image until the second time based on the second reference index; and
determining the target output speed based on a ratio between the target iteration count and the reference iteration count.

7. The electronic device of claim 1,
wherein the operation controlling the output of the reference image at the target time after the first time based on whether the first target index corresponds to the first reference index comprises:
when the first target index does not correspond to the first reference index, determining a second reference image corresponding to a target index pattern generated based on the first target index;
determining a target output speed of the second reference image based on a second target index with respect to the second time after the second time; and
controlling the second reference image at the target output speed at the target time.

8. The electronic device of claim 7,
wherein the operation controlling the output of the reference image at the target time after the first time based on whether the first target index corresponds to the first reference index further comprises:
stopping outputting the reference image when the second reference image corresponding to the target index pattern is not determined.

9. The electronic device of claim 8,
wherein the at least one processor is further configured to perform:
an operation determining a third target index generated with respect to a second motion of the user after the output of the reference image is stopped;
an operation determining a third reference image based on the third target index; and
an operation outputting the third reference image to synchronize with the second motion.

10. The electronic device of claim 3,
wherein the operation determining whether the first target index corresponds to the first reference index further comprises:
when the difference between the target index pattern and the reference index pattern exceeds the first threshold value, determining whether the difference is less than or equal to a preset second threshold value; and
when the difference is less than or equal to the second threshold value, determining that the first target index corresponds to a target range that is preset with respect to the first reference index,
wherein the operation controlling the output of the reference image at the target time after the first time based on whether the first target index corresponds to the first reference index further comprises:
an operation pausing the output of the reference image when it is determined that the first target index corresponds to the target range that is preset with respect to the first reference index.

11. The electronic device of claim 1,
wherein the at least one processor is further configured to perform:
an operation outputting the reference image on a first area of a display and outputting an additional reference image on a second area of the display for a second user to be wearing a second wearable device.

12. The electronic device of claim 11,
wherein the outputting the additional reference image on the second area of the display for the second user wearing the second wearable device comprises:
controlling an output speed of the additional reference image based on the output speed of the reference image.

13. The electronic device of claim 1,
wherein the at least one processor is further configured to perform:
obtaining a heart rate of the user; and
controlling the output of the reference image based on the heart rate.

14. The electronic device of claim 13,
wherein controlling the output of the reference image based on the heart rate comprises:
increasing the output speed of the reference image when the heart rate is lower than a lower threshold heart rate that is preset with respect to the reference image.

15. The electronic device of claim 13,
wherein controlling the output of the reference image based on the heart rate comprises:
decreasing the output speed of the reference image when the heart rate is higher than an upper threshold heart rate that is preset with respect to the reference image.

16. A method, performed by an electronic device, outputting a reference image, the method comprising:
obtaining first sensing information with respect to a first motion of a user wearing a wearable device, wherein the first sensing information is measured at a first time;
determining a first target index for the first motion based on the first sensing information;
determining a first reference index of a reference image output at the first time;
determining whether the first target index corresponds to the first reference index; and
controlling an output of the reference image at a target time after the first time based on whether the first target index corresponds to the first reference index.

17. The method of claim 16,
wherein the determining the first target index for the first motion based on the first sensing information comprises:
determining first values of one or more preset exercise indicators; and
determining the first target index based on the first values of the exercise indicators.

18. The method of claim 16,
wherein the determining whether the first target index corresponds to the first reference index comprises:
generating a target index pattern with respect to a preset target time based on the first target index;
generating a reference index pattern with respect to the target time based on the first reference index;
determining whether a difference between the target index pattern and the reference index pattern is less than or equal to a preset first threshold value; and
determining that the first target index corresponds to the first reference index when the difference is less than or equal to the first threshold value.

19. The method of claim 16,
the controlling the output of the reference image at the target time after the first time based on whether the first target index corresponds to the first reference index comprises:
when the first target index does not correspond to the first reference index, determining a second reference image corresponding to a target index pattern generated based on the first target index;
determining a target output speed of the second reference image based on a second target index with respect to the second time after the second time; and
controlling the second reference image at the target output speed at the target time.

20. The method of claim 19,
wherein the controlling the output of the reference image at the target time after the first time based on whether the first target index corresponds to the first reference index further comprises:
stopping outputting the reference image when the second reference image corresponding to the target index pattern is not determined.
